SEQUENCE LISTING (14 sequences)

SEQ ID NO: 1 - DNA, 15000 bp, Mus musculus
  source 1..15000: mol_type = genomic DNA
tctcaggggaaaggtatcctggattcagtaaccaaggaacatcacaatgtcacattgcacaacaaacctcacacaagagatttattgggagagatacaagaaggtggctgcctttccttggtggagaaacagcaggaaacggagtgggaggcaagctttcttggtggtggagttttccagggtggaaatttctagggaggggattggtaaggtttggtgagttttcaaaccctgaggtgtgtgcttggaccttacaccctacacatgcacctcatgatacaacgcccacttcccatacggagccctgtgctcaccacagtccttcttggactggctgaatttaggctcctgcataaccttgtagcccttccctcctttcctactgcaaaaccctcttagcttgctgtggtaaatacacttctgtaattcccgtaattaggaggtggaggcagggaaatagcaaagttaggtcactctagatcaagtccctcttgcaaaaacaaagaaaaaaaccacgccctctatggtacaaaccagttcctcttccctagagttctggcccctccccgggaggatgtggtcatagggtcaggtcctggggccaaggtctgtcctggtacaaccccggactgaccggtttctttcactttggtcggctctgctatgtggctcggccttgcttttgtgatggctaacgttatgatggcagttaattttatgtttaaaagtttaaaatgtctgtgcttaagaaaactataaagcgttaagagtggttaaggcttagaatttgtttttacaactattttggtttctataaatgagcttgttttgctccagaggaccctctgcttggcccagaacaaattgtaactttgtattccttgcctttataaacccttgactgaggtagctgggtgctgcattagggaatcccaacttgcaaatacggacctggatggagccagtatctgaataaaagcctcttctttgttagaatgtattaatggtcagactggcgagtctctgaatgaccccagacccataacactgtcttcacgtcctagtggcaatcaccaaaagtcccttttgaaaccaaaagttgaataagtgtcccgttgttcctccggtcttgcaaggccttgcaaagctgatcacagccttcctctcttcctggagcaccaggaacctgttatagtctgcctttattgtctataaaatgaaatcacctatgaagattccccaccctctcttccctggttgcttggctttttcttctcagactgtcccagaccagtgatagatgcccaccaacccggatcagtggtagatgcccactaacaatcccagaccctagtaagggagggtaagggggtgggggtgggggttggggctggtggcacttgcctttaatccctgcattcaggaggcagacgaaagcttatttctcagttgtagtctagagatgccatgctctggctcttgaactaagaaggtaccactggcaactctggaagcccaggagggagggagggagggagggaggaagggagggagggagggaggaagggagggagggagggagggagggagggagggagggagggagggagggttggttcctactgagcaaaataaatgctgggtagaaaaagccacagtgtcctgctccccaccttctgcccagcacttcagctgtaagacaaggcaatgccctttgatttagcacttcactctggaaagagtacaattctgagatgacagcgagatcatcatccctgtgtatttctgcctgctcccagtgacccaagttgaccgtgattgaggttattttgtaaaatatttccttgaggcaccgggaatcaaggccatgcatcctaggcgagcattctgccactgagtctcatgcttccaccgaggagccacatccccagtccttttgtaagagaatttgccgatgtaggtggccatggtggcacaggaaattagggaggctgaagcaggcaggagaatcacaattaagtcaacctgagaaacatagtgtctccagaggaaaagaatttgcaactgtgattaagggcccatattgagtctggctacacagccctgactaaccagaactcactgtgtagacagctggctggcctcgaactcatacagacctacctgcctttgcatcatggcagtaaaatcatgtaccgccatggactctcactctgtgggagtatccttgatgggcctgacccaatcggatgagccctgaaaagggaccagaagtttcctggcaaagcagacacagtgtgaagcaattcaatgcagggacattctctgctccaccctctgttttattgcttttaattcttaaaattaatttatttattctatgtacaatgttttccctcatgcatgcccatgcaccatgtgcatgcctgatacctgcagagtccagaagaggcaatcagaacccctggaattggagttatagacagctctgagccctcctgtaggtagatgggaatagagcctggatcctttggatctaaagtccatacctgctgagcgacacctccagccctgacactggctttaaagttagaggaagtaggtggccagaaatgtagaaatcacacgtagccgtggtcgacagctgagaagaagctgggacatctgtcctgcagccacatggaagtgagttctgaccaggacctggggggggatctcactttagaggctccagagacttcagcttgaacctcagcgttctgatgatctgggcagaaactctaactatgcagcaccaagtgtttacccatgggtctgtgcatccgtaaaagtagttttggggggcattgttttagggtgctcagagtgtggtaattggtcacacagcagaaggaagctgcatgaaggaaggtcacataagcattctgtaagattctgaaagcaggttttgtcccatcattcttaccttaccctcaacaggacctggaacaccatcctagtaaagtcaggaaaatggagggcaggaacctaactcttcagtccgctactcccaccagtgactccctgctcaaaacacttctacagcgctgttctgctgggtctcccatgaagcccttgctcctcagacccaggagcaagcttgaccaatctctacctctgaagcttctgagacctttgcccgcatctattgatcctcactcctgggcaggcagcggggccatcggcagacgccatgacggctctgttgttcctggggtctctgctgatgagtctggatctgacactttcggtgaggacggtggacactgggattgagccagagtggcagtgaggggtccctgttccggatttagcctctgcctgtcacctgctagttcaggtgactgagagacttgtggtgggggtgggggttgggggaaggcacagctagcccagaagatccaaacaaaggaggtagcagcccatgctggtggatgtgaggacacaggctacttttgtgggggagtcaatgagaagagaaaggaaggctacactttgtgaggaaatcctgtactttggcgaggtggcaggaggttgaggcctataggatcaagggaagtctaacattgtgggcaatgttcctgccttctctctcctataggctccaccatggaaagactccaagaaatttaaggacgcacctgatgggcccacagtgggtaagtgatctggcctcttttcctaatctactctgtggggatggctgtgctttagcctgcgtggtctcaggcctctggagcaaggagggatcctgagccaagctgttccgttgacatttaaacctgggtgcctacagattcagatgtgtgcccggagtgccaggcttgggctgtcctggatgcctgtgttagagcctggcaggtactctgtagtgttggagatgatgtcatctgatgatgtcatcacacctgcacaccaacaagtgctcgccagtttccccatcttcctccctttcctttcaacactcctggagaatgtcagacataaccggtagagttcagagcttcagtgggcgatgggagccagaacagagctttagactcacccaagatcacagacaccccaagttgtctccaaaccctaactttctcacttgtgagtttactctctgcttcatttttccctgtctgaaaagttagacgggttgctgaggagaaagggttcctcattgttgtagatgctgctgccaggaaaatgccctaacatgttagctcttaggctagggcatagctcactggcagagcacttgcccaacatgcacgcacacacacctgggtttggtctgcagcactgtgaggggaaaaaattccttttttttttaattttaaagaaaaaattctttcttaagctggagagataactcagaggttaagagcactggctgctcttccagaggtcctgagttcaattcccaacaaccacatggtggctcacaaccatctatctgtactgggatctgatgcctttttctggtctgtctgaagacagcaacacatatagtaaataattaattaattaaagaaagaacaattcttttttaaagatttatttatttattttatgtatgagtgttctatctgcatgtacacctgtatgccagaagagggtatcagatctcattacagatagctgtgagccaccatgtggttgctgggagttgaactcaggacctctacaagaacagacagtagatctcaaatgctgagccatttttccagcccaagaaaaaaaatcttaacttttagtaaatttgacttaggtgaagggttccacccccatcctgccccaaaccctgctataggacttgtgagtacagaactctaccacagtgagccttgtctgtggggtcaagctgaggcttgtagtgtgatgtctctcactgaagcccaactcagtgtgaccctccaggcttctgtcctaaccactctaagccaatcagctgctgtccctttcaacctgacccaccatggccccctgtctccaagttctaggacccttccctctctctatcaccctgttgctaaattgccactaaaaggagaaaagaagaagaagaagaagaagaagaagaagaagaagaagaagaagaagaagaagaagaggaagaaacaaaacacatttgttgagcacctactgagtcctggactcaatgtcccaagcattgccactaagcctcacggtgttcccctgaagtagacagtgatttctttttcattttctctttgagattataatataattacatcactgggcagtggtggcacatgcctttaatcctagcacttgggaggcagaggcaggcagatttctgagttcgaggccagcctgctctacacagtgagttccaggacagccagggctacacagagaaaccctgtctcgaaaacaaaaacaatatatatatatatatatatatatatatatatatacacatatatatgtgtgtgtgtgtgtgtatacgtatatatatatatacacatatatatatacacatatatatatacatatatataatcatatcatccccctccctttcttccctccaaaccctcccatagccccaataattatttctttttttttttaagatttatttatttattatatgtaagtacactgtagctgtcctcagatactccagaagagggcatcagattttgttacggatggttgtgagccaccatgtggttgctgggatttgaactcgggaccttcggaagagcagtcggcgctcttaaccactgagccatctcgccagccccaataattatttcttatgattaactatacttttctcagctaccatgaacatcaagttcacaaattcagtgattccgttcacaaattaggttccgtgagcctctggctatgacattttcaccaatcattagcatgtaatcttgttacatctggattctgtttaaagacattattcagtgtgcatgttgttgatcccttaaacactaatctaatggccgatgccactattactgcctgcctgacaaggtgtccctgacacgttttcttcataaggcgcatacaacttcttgtgttcttgcaagtaggagcctcaggtaacacttgggcatacacatagaaccactctgtctccctgaagtactgaggatcgaacctagagccttgtgtatgccaggcaaatgcttggccactgagttaaaactctggccctctttaaaaagaagtgtgtgtgtatgtgtgtgtgcaagtgcatgtgtgtatgtgtatgtacatatgtgtgtgtgtttgtgtgtgtgtatgtgtgtatatgtgtgcgtgtgtgtgtgcaagtacatgtgtgtgtatgtgtgtgtgttcgagcatctttcactgacccattcaattccataaagatcatgaaggcactgtgaacgttggcagtttctttggctttctttggccttgtgctgacttgtgagcaagcactacacgactgacctatatcctcagcctgagtcacaagtattgggttggagggggcttacaaataaatgtttacaaagcaggcaggggcagtggtgggggggtgcctttaatcccagcacttggcaggcagaggcaggtgaatttctgagtttgaggccagcctggtctacacagtgagttccaggacagccagggctacacaaagaaaccctgtcttgggggtgggggtgggggaacaaaacaaaaaacaaaacaaaacaaagcaggcaggttcacaaatataaagtcagtgaggattggcagttgcattactgttgccagtttactaagccaaaaatggccaagggacctaagtgtttgtttgtttgtttgtttgtttgtttgtttgtttgtttgttttgtagacaggctatctctgtgtaatagacctggctgtcctggaactcattctgtagaccaggctggccttgaactcacagagatccgcctgcctcagcctcccaaattctgggatgaaaggcgtgtgacctaaatctttaatgagctcaactttgtgacatgcacctgtgtttcagacttgctcagggtcagatatctattccagctgtctgactctagaagcctactttctaagcaggatgcagtcttgaatgcatagctgacctttttccaaatcaggccctgtgaggcagagggtgggcagctgggtctcagggcagggctctgaggtgtggtgttcctacagttctcactgtggatgggaggctctgccattttccctttcagtaccaccgtcagctacaccacaaatgcatccacaaaaggcggccaggctcccgcccctggtaagacattttctaataggggttgggggagggggctggatgaaatggactctgtctagctatctaagcatcgtgtttggtcaaaaggtgtgtgtgtgtgtgtgtgtgtgtgtgtgtgtgtgtttgtgtgtgtgtgtgaagcaaggctagcctagccagcctcaggcactgcagggaaacctcatctatcccccaggtgtgctaccacccccaactttgatgaagatcagcaatggggatactgcttggagcccaagaaagtgaaaggtatgtatggcatgcaggccccgggtggctcaaggctgtgtgtgtcttgctgctcactccgttcccacacacctactcacctactaagcatcatcagatgccaaacacttgggattctgggccccgcccttttccttctcttacagcattctttcaaaagtcagaggaagaattctggggaaagaggccaccattgtccagatgagcttgagggtctagaagcttggggttctatggtttgatgacaagcaggtgcaggcagggtattggtccctacatatgtccttccgactccggaagtgcttcgctcttgttatctacctctcagaccattgcagcaaacacaacccgtgccacaaaggagggacatgtatcaacacccccaatgggccacactgtctctgccctgaacacctcactgggaaacattgccagaaaggtaagaggaactgcctcccagcaagatgtccctggagacccggtgctttgccatggtcccattgacttccttgtgtccccagagaaatgctttgagcctcagcttctcaagttcttccacgagaatgagctatggtttagaacggggccaggaggtgtggccaggtgcgagtgcaaaggttctgaggctcactgcaagccggtggccagccagggtaagtgggtgtgcagggactgtggggaggagggcagagagtcaggaacccctggtagaaggctgggtgcaatgatgtacacaggtaaggctcagtttgcacctctccccaccccacccccagcctgcagcatcaatccgtgccttaatgggggcagctgcctcctcgtggaggaccacccactgtgccgttgccctacaggctacactggatatttttgcgacttgggtgagtaagaccccgtgtggaaaggcttgcggaggtggatagagagaatggaagtgaaccagagggctccaacagactcatccgccgactgcagggagccatctctctttctctagacctttgggcgacctgctatgaaggcagggggctcagctaccggggccaggctggaactacgcaatcgggtgcgccatgtcagcggtggaccgtggaggccacctaccggaacatgactgagaagcaagcgctaagctggggcctgggccaccacgcattttgccggttcgcgagaagggaccgggcaggggaacttgctttctcttagggtcctcgagggcctccccacgttctaacagtgctccctcttgagattgcaggaacccagataatgacacacgtccatggtgcttcgtctggagtggcgacaggctgagctgggactattgcggcctggagcagtgccagacgccaacgtttgcacctctagttgtccctgagagtcaggaggagtccccgtcccaggcaccatctctgtcccatgcaccaaatggttaggcagaggagggggtcccggcgcagaggacatgggtctctcttattcctggcagcccgtgccaggtatccatggcctcagccagtctctccttccacagactcgaccgatcatcagacttctctgtccaagaccaacacgatgggctgcggacagaggttccgcaagggactgtcctcgttcatgcgcgtggtgggcggactagtggctctgcctgggtcgcacccctacatcgctgcactgtactggggtaacaacttctgcgcgggcagtctcatcgccccctgttgggtgctgaccgcggctcactgcctgcagaatcggcaagtgccaccctcggtgaccccctagaccgctcctaccgtacccgcaccctactctttccctgcccgccattcttgagctccctcgaggggttggaaactaaggcacccccagagcatttgtagccggtctgagcctgctgcctgtcccccacccgactgcaggccagcgcccgaggaactgacagtggtacttggtcaagatcgccacaaccagagctgcgagtggtgccagactctggctgtgcgctcctaccgccttcacgagggcttctcctccatcacctaccagcacgacttgggtggggtggccctacagggatagggagaaaggatggcggagggctggggccctatgtcgccatctaacctttgcctctcggggtagctctgctgcgcctgcaggaaagcaaaaccaacagttgcgcgatcctgtcacctcacgttcagcctgtgtgtctacccagcggcgcggccccaccctctgagacagtgctctgcgaggtggccggctggggtcaccagttcgagggtaggcacaactgttgggcgctggttggagacttttggttatctaggagcgcagttggtacgccccgatgaatctgggggacaagtttcactgacatgacagttgtaaaaaacgcacagagcccttgtctctgtagcgtgactttcccagattctagaattctctgtcgagattccagagccccttgaggtttgttctagttttttgccttctattgtggcgttaaacaccatgaccaaatccagcttaggcaggaaaggttttatttggcttccgaggttgcagggtttcttgctgtccaccattgaggaagccacaacagggagtggaggcaggaactgaggcaggaaacggaaagggacactccttgctgcctttctcttcatggcttgttcagctttctttttaaaaaattcacttatttttattttatgtgcatgtgagtctgcctacaagcatatgtgtgcaccatatgtacatctggtacaggggaagccagatctgtaatacaggttaaaaaccacggtgtgggggctgggaacggaaccaaggccctctgcaagaacagcaagagctcttaacctctgagctagcccccaacaccttgcggcttgcttactttgctttgcttttctcttttcctcttcctcttcctcttcctcttcctcctcctcctctttctccccctcttcctcttgttttgtttttgttttttgttttgaaggcagggtttctctgtgtagtcccagctgtccgggagctgtccaggagctgtccagctctgtagaccaggctgccttcaaccttaagcactccatctgcctttgcctcccaagtgctgggattaaaggcttgcacctttctggctcagtttctttcttacacaatccaggcccacctgttcagggcggcatcactcacagtgggcggggccctttcacatcaaccattaatcaagaaaatgcccggggctggtgagatggctcagtgggtaagagcacccgactgctcttccgaaggtccagagttcaaatcccagcaaccacatggtggctcacaaccatctgtaacgagatctggcgccttcttctggagtgtctgaagacagctacagtgtacttacatataattaataaataaatctttaaaaaaaaaaaaaaagaaaatgccctacagatattgccctaggccaatcttctggaggcatctcttcgggtgaggttccttccccccctagatgactcttgagtgcgtcaaattgacagacagtaacccagcacaaggattacagggagacttgaactgtcttgtgcttcttttgggctccaaattataaaggcttaaccaggactttgtccccatgctggagcaatggagacaatggagagaactattctgggagccaggttctgagccactgtgtggaagaggataggaagtgcttcctgtgttttaagccctgctcttctctgggcttcagtgtccttgccatgaaatacttattggcaggtccctatcactcaggcttgctgtgagggagcaaagcggagtaggtggggaattgtctggtagcctggcccacgcagcaagctcaggtcctcccctctgatttgcaggggctgaagaatactccaccttcctgcaggaggcacaggttccctttatcgccctggatcgctgctccaactctaacgtgcacggagacgccattctccctgggatgctttgcgctggcttcttggagggaggcaccgatgcctgccaggtcagccctggggtcctggtaggtaccttggtccctgcctgtcaagcataaggcaagaaccacgtgctgcctgttccccacccagggtgactccgggggccctctggtgtgtgaggaaggaactgcagaacatcagctcaccctgcgcggagtcatcagctggggctccggctgtggtgaccgcaacaagcccggagtctacacagacgtggccaactacctggcttggatccagaagcatattgcttcataactaaccaggctttatccttccctccttgtgtgctccttgggatgggacgatgaatgtggcatgctgggtcacagtgaagctagtgccccgacactgggggcacagaaactcaataaagtgctttgaaaacgttcctcagaattctgtcttgaaacgtcaagtgggagcacaggtaagccaactccctcgttgcctggacaaggcaactagccagatgtcagcataagaggcgtagactcttgtccggaccaccatattttctcatccttactttgggtgagcttttgccgcccatgttcaagtccacctgaggtcaattaactggactctagggagaaggcagtcttggcatatttttaaaacgctacgggtgattccttatgcaatagaggttgggaactgtagttaagagtgttccagacgggaatcgtggtgcggatagtagctgctgagcatgtgtgagaggccctgggttagatcccagcaccaccattaaaagaagggggaggggttgtgttgggttgtgtgggttttgtttgtttgttttgaactttaagcctggcatggtggtgcaagttggtgtcaaggaaaatctgaggctgcatctgatgcctctaagtacaagctgggttaggagccagccagccagagccttcggggatgcggtgactccaaagcgaggtttgtagttcccttgcaggagaggagcctagttgtccagatgagtacatttcttgcttttaaaaatgtccttctgtttattgatttattgagatatctgtcgtcctaatgccgaaattataggcctatacaatcacccagtttatgtggtgcagggggatcaatcccaaaactttgttcttggtctagccaggccctctgtcaactgcactgtactgactgcgctacactccccgtccttacacccatctttgcacagcatcttactgcatagcttttgtctggcctgtgacttgctaggtagccttgaacttgtacagatccccttgcttcatgagtgacaggtttaagtacaagccaccatgtctggcttttttttttttttttaattaagaaaaaaaaatggggctggtgagatggctcagtgggtaagagcacccaactgctcttctgaaggtctggagttcaaatcccagcaaccacatggtggctcacaaccatccgtaatgagatctgatgccctcttctggagcgtctgaagacagctacagtgtacttacatgtaataaataaataaataaataaataaatcttaaaaaaaaaaagaaaaaaatgtggtttgtgttatgagtgtgtcacagtgtggtgtgaaggtcagagcacaactctgtggagccagttctctccttctgcctttgtgggggctctggtgataaaactcaggccaccaggcttataagacaatgcacccttactgtctgagccttttttttttttttttatggtttttcgagacaggctttctctgtgtagccctggctgtcctggaactcactttgtagaccaggctggcctcgaactcagaaatccgcccgcctctgcctcctgaatgctgggattaaaggcgtgcaccaccatgcctggcacttttttaataattaaaaacctgagaatttgggctggagagagaatgactcagtggttaagagcacggactgctcttccagaggtcctgagttcaattcccagcaatcacatgatggctcacaaccatctgtaatgggatccaatgccctcttctggtatgtctgaagacagcgacattgtactcatatacataaaataaatacatttcttttattatatctaagtacactgtagctgtcttcagatgcaccggaagagagcgtcagagctcattaaggatggttgtgagccaccatgtggttgctgggatttgaactcaggaccttcagaagagcagtcagtgctcttaaccactgagccatctctccagtcctaaatgtgtctttaaaaaaaaaaaagttaaaatttcaagacttttgagctggtgagatggctcagtgggtaagagcacccgactgctcttccgaaggtccgaagttcaaatcccagcaaccacatggtggctcacaaccacccgtaatgagatctgatgccctcttctggtgtgtctgaagacagctacagtgtacttacatataataaataaatcttaaaaaaattttttcaagacttttaaattgctgggcagtggtggcgcatgcctttaatcccagcactcggaggaagaggcaggcggatttctgagttcgaggccagcctggtctacaaagtgagttccaggacagccagggctatacagagaaaaaacaaaaaacaaaacaaaaacaaaaaaaatcaagacttttatgtgtttctgtggacatgagtgcaggtgcccaagcaagccagatgcactgctttacctgggtgctgggaattgaactctggtcctctgaaaatggcactcagttgcgggacctttttttatttatttttttattttattttattttaaatcatgtgtgtgtttgtgtgtgagcttgcctcaggagttgtcagatctctcgagtgggagttacaggctttgtaggccaaaagaggtgatgctgggaacggaactcaggctctgtgtaatgaatgctctttcttaacgctgagccacctttccagcctctcatccatcttcttccatttcctcctacatgctacttttagaaccgcccccgaacccccaccaactctaccgccctctccttctcctcctagttagatgttctgtgacgtttgccttacgtcacagccccgccctgcgaacttcgggcatgctgccagtgttcttagctctgaatgcagccggccaccccccctaatgcaagcacaagttgctgggactgactgagcggagatgagtgactggaagggctacatcagtgcagtgctgcgggatcagcggatcgatgacgtggctatcgtgggccactcggacaatcgctgtgtgtgggcatcacggcctgggggtctgctggctgccatctccccgcaggaggtgggtgtgctcaccgggccagacc

SEQ ID NO: 2 - DNA, 16 bp, synthetic construct
  source 1..16: mol_type = other DNA
agcactttattgagtt

SEQ ID NO: 3 - RNA, 23 nt, synthetic construct
  source 1..23: mol_type = other RNA
taaagcactttattgagtttctg

SEQ ID NO: 4 - RNA, 21 nt, synthetic construct
  source 1..21: mol_type = other RNA
gaaactcaataaagtgcttta

SEQ ID NO: 5 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-phosphorothioate
  modified_base 5: mod_base = OTHER; note = 5-methyl 2'-deoxycytidine 3'-phosphorothioate
  modified_base 6..8: mod_base = OTHER; note = 2'-deoxythymidine 3'-phosphorothioate
  modified_base 9: mod_base = OTHER; note = 2'-deoxyadenosine 3'-phosphorothioate
  modified_base 10..11: mod_base = OTHER; note = 2'-deoxythymidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

SEQ ID NO: 6 - RNA, 23 nt, synthetic construct
  source 1..23: mol_type = other RNA
  modified_base 1: mod_base = OTHER; note = 2'-O-methyl uridine 3'-phosphorothioate
  modified_base 2: mod_base = OTHER; note = 2'-fluoro adenosine 3'-phosphorothioate
  modified_base 3..4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-fluoro cytidine 3'-phosphodiester
  modified_base 7: mod_base = OTHER; note = 2'-O-methyl adenosine 3'-phosphodiester
  modified_base 8: mod_base = OTHER; note = 2'-O-methyl cytidine 3'-phosphodiester
  modified_base 9..11: mod_base = OTHER; note = 2'-O-methyl uridine 3'-phosphodiester
  modified_base 12: mod_base = OTHER; note = 2'-O-methyl adenosine 3'-phosphodiester
  modified_base 13: mod_base = OTHER; note = 2'-O-methyl uridine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro adenosine 3'-phosphodiester
  modified_base 17: mod_base = OTHER; note = 2'-O-methyl guanosine 3'-phosphodiester
  modified_base 18..20: mod_base = OTHER; note = 2'-O-methyl uridine 3'-phosphodiester
  modified_base 21: mod_base = OTHER; note = 2'-O-methyl cytidine 3'-phosphorothioate
  modified_base 22: mod_base = OTHER; note = 2'-O-methyl uridine 3'-phosphorothioate
  modified_base 23: mod_base = OTHER; note = 2'-O-methyl guanosine
taaagcactttattgagtttctg

SEQ ID NO: 7 - RNA, 21 nt, synthetic construct
  source 1..21: mol_type = other RNA
  modified_base 1: mod_base = OTHER; note = 2'-O-methyl guanosine 3'-phosphorothioate
  modified_base 2: mod_base = OTHER; note = 2'-O-methyl adenosine 3'-phosphorothioate
  modified_base 3..4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl uridine 3'-phosphodiester
  modified_base 7: mod_base = OTHER; note = 2'-fluoro cytidine 3'-phosphodiester
  modified_base 8: mod_base = OTHER; note = 2'-O-methyl adenosine 3'-phosphodiester
  modified_base 9: mod_base = OTHER; note = 2'-fluoro adenosine 3'-phosphodiester
  modified_base 10: mod_base = OTHER; note = 2'-fluoro uridine 3'-phosphodiester
  modified_base 11: mod_base = OTHER; note = 2'-fluoro adenosine 3'-phosphodiester
  modified_base 12..13: mod_base = OTHER; note = 2'-O-methyl adenosine 3'-phosphodiester
  modified_base 14: mod_base = OTHER; note = 2'-O-methyl guanosine 3'-phosphodiester
  modified_base 15: mod_base = OTHER; note = 2'-O-methyl uridine 3'-phosphodiester
  modified_base 16: mod_base = OTHER; note = 2'-O-methyl guanosine 3'-phosphodiester
  modified_base 17: mod_base = OTHER; note = 2'-O-methyl cytidine 3'-phosphodiester
  modified_base 18..20: mod_base = OTHER; note = 2'-O-methyl uridine 3'-phosphodiester
  modified_base 21: mod_base = OTHER; note = 2'-O-methyl adenosine phosphodiester
  modified_base >21: mod_base = OTHER; note = Trishexylamino-N-Acetylgalactosamine-hydroxyproline
gaaactcaataaagtgcttta

SEQ ID NO: 8 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-mesyl phosphoramidate
  modified_base 5: mod_base = OTHER; note = 5-methyl 2'-deoxycytidine 3'-phosphorothioate
  modified_base 6..8: mod_base = OTHER; note = 2'-deoxythymidine 3'-phosphorothioate
  modified_base 9: mod_base = OTHER; note = 2'-deoxyadenosine 3'-phosphorothioate
  modified_base 10..11: mod_base = OTHER; note = 2'-deoxythymidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

SEQ ID NO: 9 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-phosphorothioate
  modified_base 5: mod_base = OTHER; note = 5-methyl 2'-deoxycytidine 3'-phosphorothioate
  modified_base 6..8: mod_base = OTHER; note = 2'-deoxythymidine 3'-phosphorothioate
  modified_base 9: mod_base = OTHER; note = 2'-deoxyadenosine 3'-phosphorothioate
  modified_base 10..11: mod_base = OTHER; note = 2'-deoxythymidine 3'-phosphorothioate
  modified_base 12: mod_base = OTHER; note = 2'-deoxyguanosine 3'-mesyl phosphoramidate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

SEQ ID NO: 10 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-phosphorothioate
  modified_base 5: mod_base = OTHER; note = 5-methyl 2'-deoxycytidine 3'-phosphorothioate
  modified_base 6..8: mod_base = OTHER; note = 2'-deoxythymidine 3'-phosphorothioate
  modified_base 9: mod_base = OTHER; note = 2'-deoxyadenosine 3'-mesyl phosphoramidate
  modified_base 10..11: mod_base = OTHER; note = 2'-deoxythymidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

SEQ ID NO: 11 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-phosphorothioate
  modified_base 5: mod_base = OTHER; note = 5-methyl 2'-deoxycytidine 3'-phosphorothioate
  modified_base 6..7: mod_base = OTHER; note = 2'-deoxythymidine 3'-phosphorothioate
  modified_base 8: mod_base = OTHER; note = 2'-deoxythymidine 3'-mesyl phosphoramidate
  modified_base 9: mod_base = OTHER; note = 2'-deoxyadenosine 3'-phosphorothioate
  modified_base 10..11: mod_base = OTHER; note = 2'-deoxythymidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

SEQ ID NO: 12 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-phosphorothioate
  modified_base 5: mod_base = OTHER; note = 5-methyl 2'-deoxycytidine 3'-phosphorothioate
  modified_base 6..7: mod_base = OTHER; note = 2'-deoxythymidine 3'-phosphorothioate
  modified_base 8: mod_base = OTHER; note = 2'-deoxythymidine 3'-mesyl phosphoramidate
  modified_base 9: mod_base = OTHER; note = 2'-deoxyadenosine 3'-mesyl phosphoramidate
  modified_base 10..11: mod_base = OTHER; note = 2'-deoxythymidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

SEQ ID NO: 13 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-mesyl phosphoramidate
  modified_base 5: mod_base = OTHER; note = 5-methyl 2'-deoxycytidine 3'-phosphorothioate
  modified_base 6..7: mod_base = OTHER; note = 2'-deoxythymidine 3'-phosphorothioate
  modified_base 8: mod_base = OTHER; note = 2'-deoxythymidine 3'-mesyl phosphoramidate
  modified_base 9: mod_base = OTHER; note = 2'-deoxyadenosine 3'-mesyl phosphoramidate
  modified_base 10..11: mod_base = OTHER; note = 2'-deoxythymidine 3'-phosphorothioate
  modified_base 12: mod_base = OTHER; note = 2'-deoxyguanosine 3'-mesyl phosphoramidate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

SEQ ID NO: 14 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl guanosine 3'-phosphorothioate
  modified_base 3: mod_base = OTHER; note = constrained ethyl 5-methylcytidine 3'-phosphorothioatee
  modified_base 4: mod_base = OTHER; note = 2'-deoxyadenosine 3'-mesyl phosphoramidate
  modified_base 5: mod_base = OTHER; note = 5-methyl 2'-deoxycytidine 3'-phosphorothioate
  modified_base 6..7: mod_base = OTHER; note = 2'-deoxythymidine 3'-phosphorothioate
  modified_base 8: mod_base = OTHER; note = 2'-deoxythymidine 3'-mesyl phosphoramidate
  modified_base 9: mod_base = OTHER; note = 2'-deoxyadenosine 3'-mesyl phosphoramidate
  modified_base 10: mod_base = OTHER; note = 2'-deoxythymidine 3'-phosphorothioate
  modified_base 11: mod_base = OTHER; note = 2'-deoxythymidine 3'-mesyl phosphoramidate
  modified_base 12: mod_base = OTHER; note = 2'-deoxyguanosine 3'-mesyl phosphoramidate
  modified_base 13: mod_base = OTHER; note = 2'-deoxyadenosine 3'-phosphorothioate
  modified_base 14: mod_base = OTHER; note = constrained ethyl guanosine 3'-phosphorothioate
  modified_base 15: mod_base = OTHER; note = constrained ethyl thymidine 3'-phosphorothioate
  modified_base 16: mod_base = OTHER; note = constrained ethyl thymidine
agcactttattgagtt

(19) United States
(12) Patent Application Publication
(10) Pub. No.: US 2024/0360448 A1
(43) Pub. Date: Oct. 31, 2024

(54) LINKAGE MODIFIED OLIGOMERIC COMPOUNDS AND USES THEREOF

(71) Applicant: Ionis Pharmaceuticals, Inc., Carlsbad, CA (US)

(72) Inventors: Hans-Joachim Josef Gaus, Vista, CA (US); Punit P. Seth, Carlsbad, CA (US)

(73) Assignee: Ionis Pharmaceuticals, Inc., Carlsbad, CA (US)

(21) Appl. No.: 18/684,147

(22) PCT Filed: Aug. 17, 2022
(86) PCT No.: PCT/US2022/075073

§ 371 Date: Feb. 15, 2024

Related U.S. Application Data
(60) Provisional application No. 63/234,639, filed on Aug. 18, 2021.

(51) Int. Cl. C12N 15/113

(52) U.S. Cl. CPC C12N 15/113; C12N 2310/11; C12N 2310/14; C12N 2310/3145; C12N 2310/315; C12N 2310/321; C12N 2310/322; C12N 2310/3231; C12N 2310/345; C12N 2310/346; C12N 2310/3515

(57) ABSTRACT

The present disclosure provides oligomeric compounds (including oligomeric compounds that are antisense agents or portions thereof) comprising a modified oligonucleotide having at least one modified intemucleoside linkage. In some embodiments, the disclosure provides oligomeric compounds and modified oligonucleotides comprising a central region in which purine-containing central region nucleosides are adjacent to methanesulfonyl phosphoramidate intemucleoside linkages.

### SEQUENCE LISTING

The present application is being filed along with a Sequence Listing in electronic format. The Sequence Listing is provided as a file entitled CHEM0104WOSEQ_ST26.xml created Aug. 16, 2022, which is 113 kb in size. The information in the electronic format of the sequence listing is incorporated herein by reference in its entirety.

### FIELD

The present disclosure provides oligomeric compounds (including oligomeric compounds that are antisense agenst or portions thereof) comprising a modified oligonucleotide having at least one modified internucleoside linkage.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, tolerability, pharmacokinetics, or affinity for a target nucleic acid.

### SUMMARY

In certain embodiments, the present disclosure provides oligomeric compounds (including oligomeric compounds that are antisense agents or portions thereof) comprising modified oligonucleotides consisting of linked nucleosides wherein at least one internucleoside linkage is a sulfonyl phosphoramidate internucleoside linkage, for example the internucleoside linkage of Formula I:

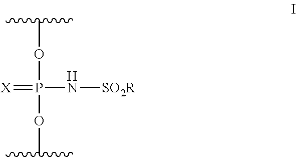

wherein:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.

In certain embodiments, the present disclosure provides methods for administering doses of such oligomeric compounds to a subject.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′—OH(H) sugar moiety and a thymine base could be described as a DNA having a modified sugar (2′—OH in place of one 2′—H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” encompasses any modified oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and modified oligonucleotides having other modified nucleobases, such as “AT^{m}CGAUCG,” wherein ^{m}C indicates a cytosine base comprising a methyl group at the 5-position.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to introducing a compound or composition provided herein to a subject to perform its intended function. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense oligonucleotide to its target nucleic acid. In certain embodiments, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an antisense oligonucleotide or an oligonucleotide duplex comprising an antisense oligonucleotide.

As used herein, “antisense compound” means an antisense oligonucleotide or an oligonucleotide duplex comprising an antisense oligonucleotide.

As used herein, “antisense oligonucleotide” means an oligonucleotide that is complementary to a target nucleic acid and is capable of achieving at least one antisense activity. Antisense oligonucleotides include but are not limited to RNAi antisense modified oligonucleotides and RNase H antisense modified oligonucleotides. In certain embodiments, an antisense oligonucleotide is paired with a sense oligonucleotide to form an oligonucleotide duplex. In certain embodiments, an antisense oligonucleotide is unpaired and is a single-stranded antisense oligonucleotide. In certain embodiments, an antisense oligonucleotide comprises a conjugate group.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “cEt” or “constrained ethyl” or “cEt sugar moiety” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′—CH(CH₃)—O—2′, and the methyl group of the bridge is in the S configuration. A cEt bicyclic sugar moiety is in the β-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms consisting of a conjugate moiety and a conjugate linker.

As used herein, “conjugate moiety” means a group of atoms that, when attached to a molecule modifies one or more properties of that molecule, including but not limited to pharmacodynamics, pharmacokinetics, stability, binding, absorption, tissue distribution, cellular distribution, cellular uptake, charge and clearance.

As used herein, “conjugate linker” means a group of atoms comprising at least one bond that links a conjugate moiety to a molecule.

As used herein, “deoxy region” means a region of 5-12 contiguous nucleotides, wherein at least 70% of the nucleosides are stereo-standard DNA nucleosides. In certain embodiments, each nucleoside is selected from a stereo-standard DNA nucleoside (a nucleoside comprising a β-D-2′-deoxyribosyl sugar moiety), a stereo-non-standard nucleoside of Formula V-XI, a bicyclic nucleoside, and a substituted stereo-standard nucleoside. In certain embodiments, a deoxy region supports RNase H activity. In certain embodiments, a deoxy region is the gap of a gapmer.

As used herein, “deoxy nucleoside” or “deoxynucleoside” means a stereo standard or stereo non standard DNA nucleoside.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region that supports RNase H cleavage positioned between a 5′-region and a 3′-region. Herein, the nucleosides of the 5′-region and 3′-region each comprise a 2′-substituted furanosyl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions. In certain embodiments, the central region encompasses internucleoside linkages that link only deoxynucleosides, and internucleoside linkages that link a deoxynucleoside to a modified nucleoside are not within the central region.

As used herein, “hepatotoxic” in the context of a mouse means a plasma ALT level that is above 300 units per liter. Hepatotoxicity of an oligomeric compound or parent oligomeric compound that is administered to a mouse is determined by measuring the plasma ALT level of the mouse 24 hours to 2 weeks following at least one dose of 1-150 mg/kg of the compound.

As used herein, “hepatotoxic” in the context of a human means a plasma ALT level that is above 150 units per liter. Hepatotoxicity of an oligomeric compound or parent oligomeric compound that is administered to a human is determined by measuring the plasma ALT level of the human 24 hours to 2 weeks following at least one dose of 10-300 mg of the compound.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, “internucleoside linkage” means a group or bond that forms a covalent linkage between adjacent nucleosides in an oligonucleotide. As used herein “modified internucleoside linkage” means any internucleoside linkage other than a naturally occurring, phosphodiester internucleoside linkage. A “neutral internucleoside linkage” is a modified internucleoside linkage that does not have a negatively charged phosphate in a buffered aqueous solution at pH=7.0. A modified internucleoside linkage may optionally comprise a conjugate group.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means O-methoxyethyl. “2′-MOE” or “2′-O-methoxyethyl” means a 2′-OCH₂CH₂OCH₃ group at the 2′-position of a fumnosyl ring. In certain embodiments, the 2′-OCH₂CH₂OCH₃ group is in place of the 2′—OH group of a ribosyl ring or in place of a 2′-H in a 2′-deoxyribosyl ring. A “2′-MOE sugar moiety” is a sugar moiety with a 2′-OCH₂CH₂OCH₃ group in place of the 2′—OH group of a fumnosyl sugar moiety. Unless otherwise indicated, a 2′-MOE sugar moiety is in the β-D ribosyl configuration.

As used herein, a “2′-OMe sugar moiety” is a sugar moiety with a 2′-OCH₃ group in place of the 2′—OH group of a furanosyl sugar moiety. Unless otherwise indicated, a 2′-OMe sugar moiety is in the β-D ribosyl configuration and is a “stereo-standard 2′OMe sugar moiety”.

As used herein, a “2′-F sugar moiety” is a sugar moiety with a 2′—F group in place of the 2′—OH group of a furanosyl sugar moiety. Unless otherwise indicated, a 2′-F sugar moiety is in the β-D ribosyl configuration and is a “stereo-standard 2′-F sugar moiety”.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. As used herein, “purine nucleoside” means a nucleoside comprising a purine nucleobase (e.g., adenine or guanine) and a sugar moiety. A modified nucleoside may comprise a conjugate group.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be attached to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-80 linked nucleosides, and optionally a conjugate group or terminal group. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds (including oligomeric compounds that are antisense agents or portions thereof), i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “phosphodiester linkage” means the linking group having the following structure:

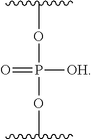 [SMILES: *OP(=O)(O)O*]

In certain embodiments, a phosphodiester linkage is an internucleoside linkage. In certain embodiments, a phosphodiester linkage links a conjugate moiety to a modified oligonucleotide.

As used herein, “phosphorothioate linkage” means the linking group having the following structure:

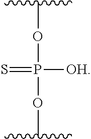 [SMILES: *OP(O)(=S)O*]

In certain embodiments, a phosphorothioate linkage is an internucleoside linkage. In certain embodiments, a phosphorothioate linkage links a conjugate moiety to a modified oligonucleotide.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNA), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNAi oligonucleotide” means an RNAi antisense modified oligonucleotide or a RNAi sense modified oligonucleotide.

As used herein, “RNAi antisense modified oligonucleotide” means an oligonucleotide comprising a region that is complementary to a target sequence, and which includes at least one chemical modification suitable for RNAi.

As used herein, “RNAi antisense oligomeric compound” means a single-stranded oligomeric compound comprising a region that is complementary to a target sequence, and which includes at least one chemical modification suitable for RNAi.

As used herein, “RNAi sense modified oligonucleotide” means an oligonucleotide comprising a region that is complementary to a region of an RNAi antisense modified oligonucleotide, and which is capable of forming a duplex with such RNAi antisense modified oligonucleotide.

As used herein, “RNAi sense oligomeric compound” means a single-stranded oligomeric compound comprising a region that is complementary to a region of an RNAi antisense modified oligonucleotide and/or an RNAi antisense oligomeric compound, and which is capable of forming a duplex with such RNAi antisense modified oligonucleotide and/or RNAi antisense oligomeric compound.

A duplex formed by an RNAi antisense modified oligonucleotide and/or an RNAi antisense oligomeric compound with a RNAi sense modified oligonucleotide and/or an RNAi sense oligomeric compound is referred to as a double-stranded RNAi agent (dsRNAi) or a short interfering RNA (siRNA).

As used herein, “RNase H agent” means an antisense agent that acts, at least in part, through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount or activity of a target nucleic acid. The term RNase H agent excludes antisense agents that act principally through RISC/Ago2.

As used herein, “RNase H antisense modified oligonucleotide” means an oligonucleotide comprising a region that is complementary to a target sequence, and which includes at least one chemical modification suitable for RNase H-mediated nucleic acid reduction.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates and 5′-cyclopropyl phosphonate.

As used herein, “stereo-standard nucleoside” means a nucleoside comprising a non-bicyclic furanosyl sugar moiety having the configuration of naturally occurring DNA and RNA as shown below. A “stereo-standard DNA nucleoside” is a nucleoside comprising a β-D-2′-deoxyribosyl sugar moiety. A “stereo-standard RNA nucleoside” is a nucleoside comprising a β-D-ribosyl sugar moiety. A “substituted stereo-standard nucleoside” is a stereo-standard nucleoside other than a stereo-standard DNA or stereo-standard RNA nucleoside. In certain embodiments, R₁ is a 2′-substituent and R₂-R₅ are each H. In certain embodiments, the 2′-substituent is selected from OMe, F, OCH₂CH₂OCH₃, O-alkyl, SMe, or NMA. In certain embodiments, R₁-R₄ are H and R₅ is a 5′-substituent selected from methyl, allyl, or ethyl. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

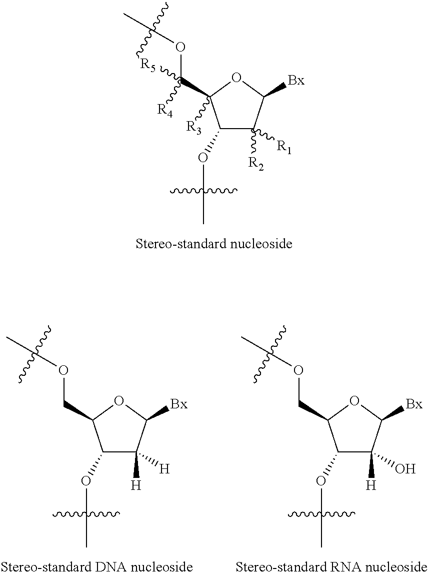

As used herein, “stereo-non-standard nucleoside” means a nucleoside comprising a non-bicyclic furanosyl sugar moiety having a configuration other than that of a stereo-standard sugar moiety. In certain embodiments, a “stereo-non-standard nucleoside” is a 2′-β-L-deoxyribosyl sugar moiety, 2′-α-D-deoxyribosyl sugar moiety, 2′-α-L-deoxyribosyl sugar moiety, a 2′-β-D-deoxyxylosyl sugar moiety, a 2′-β-L-deoxyxylosyl sugar moiety, a 2′-α-D-deoxyxylosyl sugar moiety, a 2′-α-L-deoxyxylosyl sugar moiety, a 2′-fluoro-β-D-arabinosyl sugar moiety, a 2′-fluoro-$-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, a 2′-fluoro-α-L-ribosyl sugar moiety, a 2′-fluoro-β-L-xylosyl sugar moiety, a 2′-fluoro-α-L-arabinosyl sugar moiety, a 2′-fluoro-α-L-xylosyl sugar moiety, a 2′-fluoro-β-L-ribosyl sugar moiety, a 2′-fluoro-β-L-arabinosyl sugar moiety, a 2′-fluoro-β-D-lyxosyl sugar moiety, a 2′-fluoro-α-D-lyxosyl sugar moiety, a 2′-fluoro-α-L-lyxosyl sugar moiety, a 2′-fluoro-β-L-lyxosyl sugar moiety, a 2′-O-methyl-β-D-arabinosyl sugar moiety, a 2′-O-methyl-β-D-xylosyl sugar moiety, a 2′-O-methyl-α-D-ribosyl sugar moiety, a 2′-O-methyl-α-D-arabinosyl sugar moiety, a 2′-O-methyl-α-D-xylosyl sugar moiety, a 2′-O-methyl-α-L-ribosyl sugar moiety, a 2′-O-methyl-β-L-xylosyl sugar moiety, a 2′-O-methyl-α-L-arabinosyl sugar moiety, a 2′-O-methyl-α-L-xylosyl sugar moiety, a 2′-O-methyl-β-L-ribosyl sugar moiety, a 2′-O-methyl-β-L-arabinosyl sugar moiety, a 2′-O-methyl-β-D-lyxosyl sugar moiety, a 2′-O-methyl-α-D-lyxosyl sugar moiety, a 2′-O-methyl-α-L-lyxosyl sugar moiety, or a 2′-O-methyl-β-L-lyxosyl sugar moiety.

As used herein, “stereo-standard sugar moiety” means the sugar moiety of a stereo-standard nucleoside.

As used herein, “stereo-non-standard sugar moiety” means the sugar moiety of a stereo-non-standard nucleoside.

As used herein, “substituted stereo-non-standard nucleoside” means a stereo-non-standard nucleoside comprising a substituent other than the substituent corresponding to natural RNA or DNA. In certain embodiments, a substituted stereo-non-standard nucleoside is a a 2′-fluoro-β-D-arabinosyl sugar moiety, a 2′-fluoro-β-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, a 2′-fluoro-α-L-ribosyl sugar moiety, a 2′-fluoro-β-L-xylosyl sugar moiety, a 2′-fluoro-α-L-arabinosyl sugar moiety, a 2′-fluoro-α-L-xylosyl sugar moiety, a 2′-fluoro-β-L-ribosyl sugar moiety, a 2′-fluoro-β-L-arabinosyl sugar moiety, a 2′-fluoro-β-D-lyxosyl sugar moiety, a 2′-fluoro-α-D-lyxosyl sugar moiety, a 2′-fluoro-α-L-lyxosyl sugar moiety, a 2′-fluoro-β-L-lyxosyl sugar moiety, a 2′-O-methyl-β-D-arabinosyl sugar moiety, a 2′-O-methyl-β-D-xylosyl sugar moiety, a 2′-O-methyl-α-D-ribosyl sugar moiety, a 2′-O-methyl-α-D-arabinosyl sugar moiety, a 2′-O-methyl-α-D-xylosyl sugar moiety, a 2′-O-methyl-α-L-ribosyl sugar moiety, a 2′-O-methyl-β-L-xylosyl sugar moiety, a 2′-O-methyl-α-L-arabinosyl sugar moiety, a 2′-O-methyl-α-L-xylosyl sugar moiety, a 2′-O-methyl-β-L-ribosyl sugar moiety, a 2′-O-methyl-β-L-arabinosyl sugar moiety, a 2′-O-methyl-β-D-lyxosyl sugar moiety, a 2′-O-methyl-α-D-lyxosyl sugar moiety, a 2′-O-methyl-α-L-lyxosyl sugar moiety, or a 2′-O-methyl-β-L-lyxosyl sugar moiety.p

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “sulfonyl phosphoramidate internucleoside linkage” means a modified internucleoside linkage of Formula I-IV. As used herein “mesyl phosphoramidate internucleoside linkage” or “methansulfonyl phosphoramidate internucleoside linkage” means a linkage of Formula II.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to effect an alteration or improvement of a disease, disorder, or condition in the animal.

### Certain Embodiments

The present disclosure provides the following non-limiting embodiments:

Embodiment 1. An oligomeric compound comprising a modified oligonucleotide consisting of 12-30 linked nucleosides, wherein the modified oligonucleotide comprises:
a 5′-region consisting of 2-5 linked 5′-region nucleosides, wherein the 3′-most 5′-region nucleoside comprises a modified sugar moiety;
a 3′-region consisting of 2-5 linked 3′-region nucleosides, wherein the 5′-most 3′-region nucleoside comprises a modified sugar moiety;
a central region between the 5′-region and 3′-region consisting of 8-12 linked central-region nucleosides, wherein
   each central region nucleoside is independently selected from a nucleoside comprising a modified sugar moiety and a deoxynucleoside,
   wherein the 5′-most central-region nucleoside is a deoxynucleoside and the 3′-most central region nucleoside is a deoxynucleoside and not more than 2 central region nucleosides comprise a modified sugar moiety; and
   wherein at least 1 internucleoside linkage within the central region is a sulfonyl phosphoramidate linkage.

Embodiment 2. The oligomeric compound of embodiment 1, wherein each 5′-region nucleoside comprises a modified sugar moiety.

Embodiment 3. The oligomeric compound any of embodiments 1 or 2, wherein each 3′-region comprises a modified sugar moiety.

Embodiment 4. The oligomeric compound of any of embodiments 1-3, wherein each central-region nucleoside is a deoxynucleoside.

Embodiment 5. The oligomeric compound of any of embodiments 1-4, wherein each deoxynucleoside comprises a 2′-β-D-deoxyribosyl sugar moiety.

Embodiment 6. The oligomeric compound of any of embodiments 1-5, wherein at least one 5′-region nucleoside and/or at least one 3′-region nucleoside comprises a bicyclic sugar moiety.

Embodiment 7. The oligomeric compound of embodiment 6, wherein each bicyclic sugar moiety is selected from cEt or LNA.

Embodiment 8. The oligomeric compound of any of embodiments 1-6, wherein at least one 5′-region nucleoside and/or at least one 3′-region nucleoside comprises a 2′-substituted sugar moiety.

Embodiment 9. The oligomeric compound of any of embodiments 1-3 or 5-8, wherein at least one central region nucleoside comprises a 2′-substituted sugar moiety.

Embodiment 10. The oligomeric compound of any of embodiments 8-9 wherein the 2′-substituent of each 2′-substituted sugar moiety is selected from OCH₃, and O(CH₂)₂—OCH₃.

Embodiment 11. The oligomeric compound of any of embodiments 1-10, wherein the modified oligonucleotide comprises at least one modified nucleoside comprising a modified nucleobase.

Embodiment 12. The oligomeric compound of embodiment 11, wherein the modified nucleobase is 5-methyl cytosine.

Embodiment 13. The oligomeric compound of any of embodiments 1-12, comprising a conjugate group.

Embodiment 14. The oligomeric compound of embodiment 13, wherein the conjugate group comprises a cell targeting moiety.

Embodiment 15. The oligomeric compound of any of embodiments 1-14, wherein exactly 2 of the internucleoside linkages within the central region are sulfonylphosphoramidate internucleoside linkages.

Embodiment 16. The oligomeric compound of any of embodiments 1-14, wherein exactly 3 of the internucleoside linkages within the central region are sulfonylphosphoramidate internucleoside linkages.

Embodiment 17. The oligomeric compound of any of embodiments 1-14, wherein exactly 4 of the internucleoside linkages within the central region are sulfonylphosphoramidate internucleoside linkages.

Embodiment 18. The oligomeric compound of any of embodiments 1-14, wherein exactly 5 of the internucleoside linkages within the central region are sulfonylphosphoramidate internucleoside linkages.

Embodiment 19. The oligomeric compound of any of embodiments 1-14, wherein exactly 6 of the internucleoside linkages within the central region are sulfonylphosphoramidate internucleoside linkages.

Embodiment 20. The oligomeric compound of any of embodiments 1-14, wherein exactly 7 of the internucleoside linkages within the central region are sulfonylphosphoramidate internucleoside linkages.

Embodiment 21. The oligomeric compound of any of embodiments 1-20, wherein 3 consecutive internucleoside linkages within the central region are sulfonylphosphoramidate internucleoside linkages.

Embodiment 22. The oligomeric compound of any of embodiments 1-14 or 17-21, wherein 4 consecutive internucleoside linkages within the central region are sulfonylphosphoramidate internucleoside linkages.

Embodiment 23. The oligomeric compound of any of embodiments 1-14 or 18-21, wherein 5 consecutive internucleoside linkages within the central region are sulfonylphosphoramidate internucleoside linkages.

Embodiment 24. The oligomeric compound of any of embodiments 1-23, wherein each internucleoside linkage within the central region that is immediately 5′ of a purine nucleoside is a sulfonylphosphoramidate internucleoside linkage.

Embodiment 25. The oligomeric compound of any of embodiments 1-24, wherein each internucleoside linkage within the central region that is immediately 3′ of a purine nucleoside is a sulfonylphosphoramidate internucleoside linkage.

Embodiment 26. The oligomeric compound of embodiment 24 or 25, wherein each internucleoside linkage that is not immediately 3′ or 5′ of a purine nucleoside is a phosphorothioate internucleoside linkage.

Embodiment 27. The oligomeric compound of any of embodiments 24-26, wherein 5 nucleosides within the central region are purine nucleosides.

Embodiment 28. The oligomeric compound of any of embodiments 1-27, wherein each internucleoside linkage within the central region that is immediately 5′ of an adenine nucleoside is a sulfonylphosphoramidate internucleoside linkage.

Embodiment 29. The oligomeric compound of any of embodiments 1-28, wherein each internucleoside linkage within the central region that is immediately 3′ of an adenine nucleoside is a sulfonylphosphoramidate internucleoside linkage.

Embodiment 30. The oligomeric compound of embodiment 28 or 29, wherein each internucleoside linkage that is not immediately 3′ or 5′ of an adenine nucleoside is a phosphorothioate internucleoside linkage.

Embodiment 31. The oligomeric compound of any of embodiments 28-30, wherein 4 nucleosides within the central region are adenine nucleosides.

Embodiment 32. The oligomeric compound of any of embodiments 1-29, wherein each internucleoside linkage within the central region that is immediately 5′ of a guanine nucleoside is a sulfonylphosphoramidate internucleoside linkage.

Embodiment 33. The oligomeric compound of any of embodiments 1-29 or 32, wherein each internucleoside linkage within the central region that is immediately 3′ of a guanine nucleoside is a sulfonylphosphoramidate internucleoside linkage.

Embodiment 34. The oligomeric compound of embodiment 32 or 33, wherein each internucleoside linkage that is not immediately 3′ or 5′ of a guanine nucleoside is a phosphorothioate internucleoside linkage.

Embodiment 35. The oligomeric compound of any of embodiments 1-34, wherein each sulfonylphosphoramidate internucleoside linkage is immediately 5′ or 3′ of a purine nucleoside.

Embodiment 36. The oligomeric compound of any of embodiments 1-35, wherein each purine in the central region is linked to an adjacent nucleoside by a sulfonylphosphoramidate internucleoside linkage at the 3′-side of the purine nucleoside, the 5′-side of the purine nucleoside, or both the 3′-side and the 5′-side of the purine nucleoside, independently for each purine nucleoside in the central region.

Embodiment 37. The oligomeric compound of any of embodiments 1-35, wherein the internucleoside linkage motif of the central region alternates between sulfonylphosphoramidate internucleoside linkages and phosphorothioate linkages.

Embodiment 38. The oligomeric compound of any of embodiments 1-37, wherein each internucleoside linkage within the central region is selected from a sulfonylphosphoramidate internucleoside linkage and a phosphorothioate internucleoside linkage.

Embodiment 39. The oligomeric compound of any of embodiments 1-38, wherein each internucleoside linkage within the 5′-region is selected from a phosphodiester internucleoside linkage and a phosphorothioate internucleoside linkage.

Embodiment 40. The oligomeric compound of any of embodiments 1-39, wherein each internucleoside linkage within the 3′-region is selected from a phosphodiester internucleoside linkage and a phosphorothioate internucleoside linkage.

Embodiment 41. The oligomeric compound of any of embodiments 1-40, wherein the internucleoside linkage between the 5′-region and the central region is selected from a phosphodiester internucleoside linkage and a phosphorothioate internucleoside linkage.

Embodiment 42. The oligomeric compound of any of embodiments 1-41, wherein the internucleoside linkage between the 5′-region and the central region is a phosphodiester internucleoside linkage.

Embodiment 43. The oligomeric compound of any of embodiments 1-42, wherein the internucleoside linkage between the 3′-region and the central region is selected from a phosphodiester internucleoside linkage and a phosphorothioate internucleoside linkage.

Embodiment 44. The oligomeric compound of any of embodiments 1-43, wherein the internucleoside linkage between the 3′-region and the central region is a phosphodiester internucleoside linkage.

Embodiment 45. The oligomeric compound of any of embodiments 1-44, wherein each internucleoside linkage of the modified oligonucleotide is selected from among a sulfonyl phosphoramidate, a phosphodiester and a phosphorothioate internucleoside linkage.

Embodiment 46. The oligomeric compound of any of embodiments 1-45, wherein the sulfonyl phosphoramidate linkage is a mesyl phosphoramidate linkage.

Embodiment 47. The oligomeric compound of any of embodiment 1-46, wherein the central region comprises an internucleoside linkage motif (5′ to 3′) of: zzzz; szzzz; szzzzs; or zzzzs, wherein each “z” represents a mesylphosphoramidate internucleoside linkage and each “s” represents a phosphorothioate internucleoside linkage.

Embodiment 48. The oligomeric compound of any of embodiment 1-46, wherein the central region comprises an internucleoside linkage motif (5′ to 3′) of: zszszszs or szszszsz, wherein each “z” represents a mesylphosphoramidate internucleoside linkage and each “s” represents a phosphorothioate internucleoside linkage.

Embodiment 49. The oligomeric compound of any of embodiments 1-48, wherein at least one sulfonyl phosphoramidate linkage is according to Formula I.

Embodiment 50. The oligomeric compound of any of embodiments 1-49, wherein at least one sulfonyl phosphoramidate linkage is according to Formula II.

Embodiment 51. The oligomeric compound of any of embodiments 1-50, wherein at least one sulfonyl phosphoramidate linkage is according to Formula III.

Embodiment 52. The oligomeric compound of any of embodiments 1-51, wherein at least one sulfonyl phosphoramidate linkage is according to Formula IV.

Embodiment 53. The oligomeric compound of any of embodiments 1-48, wherein each sulfonyl phosphoramidate linkage is according to Formula I.

Embodiment 54. The oligomeric compound of any of embodiments 1-49, wherein each sulfonyl phosphoramidate linkage is according to Formula II.

Embodiment 55. The oligomeric compound of any of embodiments 1-50, wherein each sulfonyl phosphoramidate linkage is according to Formula III.

Embodiment 56. The oligomeric compound of any of embodiments 1-51, wherein each sulfonyl phosphoramidate linkage is according to Formula IV.

Embodiment 57. The oligomeric compound of any of embodiments 1-56, wherein the 5′-region is at the 5′ terminal end of the modified oligonucleotide and the 3′-region is at the 3′ terminal end of the modified oligonucleotide.

Embodiment 58. A method comprising administering an oligomeric compound of any of embodiments 1-57 to an animal.

Embodiment 59. The method of embodiment 58 wherein at least two doses of the oligomeric compound of any of embodiments 1-57 are administered to the animal and the oligomeric compound is administered to the animal at a dose frequency of once per 4 months, 5 months, 6 months, 7 months, 8 months, 9 months, 10 months, 11 months, or a year or more than a year.

Embodiment 60. The method of claim 58 or 59, wherein the oligomeric compound is dosed at an amount of 50 mg to 500 mg.

Embodiment 61. The method of embodiment 58 or 59, wherein the oligomeric compound is administered systemically.

Embodiment 62. An oligomeric compound comprising a modified oligonucleotide consisting of 15-25 linked nucleosides, wherein the modified oligonucleotide comprises:
a 5′-region consisting of 2-5 linked 5′-region nucleosides, wherein the 3′-most 5′-region nucleoside comprises a modified sugar moiety;
a 3′-region consisting of 2-5 linked 3′-region nucleosides, wherein the 5′-most 3′-region nucleoside comprises a modified sugar moiety;
a central region between the 5′-region and 3′-region consisting of 8-12 linked central-region nucleosides, wherein
   each central region nucleoside is independently selected from a nucleoside comprising a modified sugar moiety and a deoxynucleoside,
   wherein the 5′-most central-region nucleoside is a deoxynucleoside and the 3′-most central region nucleoside is a deoxynucleoside and not more than 2 central region nucleosides comprise a modified sugar moiety; and
   wherein 4 consecutive internucleoside linkages within the central region are mesylphosphoramidate internucleoside linkages and the remaining internucleoside linkages within the central region are phosphorothioate internucleoside linkages.

Embodiment 63. An oligomeric compound comprising a modified oligonucleotide consisting of 15-25 linked nucleosides, wherein the modified oligonucleotide comprises:
a 5′-region consisting of 2-5 linked 5′-region nucleosides, wherein the 3′-most 5′-region nucleoside comprises a modified sugar moiety;
a 3′-region consisting of 2-5 linked 3′-region nucleosides, wherein the 5′-most 3′-region nucleoside comprises a modified sugar moiety;
a central region between the 5′-region and 3′-region consisting of 8-12 linked central-region nucleosides, wherein
   each central region nucleoside is independently selected from a nucleoside comprising a modified sugar moiety and a deoxynucleoside,
   wherein the 5′-most central-region nucleoside is a deoxynucleoside and the 3′-most central region nucleoside is a deoxynucleoside and not more than 2 central region nucleosides comprise a modified sugar moiety; and
   wherein the internucleoside linkages within the central region alternate between mesylphosphoramidate linkages and phosphorothioate internucleoside linkages.

Embodiment 64. An oligomeric compound comprising a modified oligonucleotide consisting of 15-25 linked nucleosides, wherein the modified oligonucleotide comprises:
a 5′-region consisting of 2-5 linked 5′-region nucleosides, wherein the 3′-most 5′-region nucleoside comprises a modified sugar moiety;
a 3′-region consisting of 2-5 linked 3′-region nucleosides, wherein the 5′-most 3′-region nucleoside comprises a modified sugar moiety;
a central region between the 5′-region and 3′-region consisting of 8-12 linked central-region nucleosides, wherein
   each central region nucleoside is independently selected from a nucleoside comprising a modified sugar moiety and a deoxynucleoside,
   wherein the 5′-most central-region nucleoside is a deoxynucleoside and the 3′-most central region nucleoside is a deoxynucleoside and not more than 2 central region nucleosides comprise a modified sugar moiety; and
   wherein each internucleoside linkage within the central region that is adjacent to a purine is a mesylphosphoramidate internucleoside linkage and the remaining internucleoside linkages of the central region are phosphorothioate internucleoside linkages.

Embodiment 65. The oligomeric compound of embodiment 64, wherein 5 nucleosides within the central region are purine nucleosides.

Embodiment 66. An oligomeric compound comprising a modified oligonucleotide consisting of 15-25 linked nucleosides, wherein the modified oligonucleotide comprises:
a 5′-region consisting of 2-5 linked 5′-region nucleosides, wherein the 3′-most 5′-region nucleoside comprises a modified sugar moiety;
a 3′-region consisting of 2-5 linked 3′-region nucleosides, wherein the 5′-most 3′-region nucleoside comprises a modified sugar moiety;
a central region between the 5′-region and 3′-region consisting of 8-12 linked central-region nucleosides, wherein
   each central region nucleoside is independently selected from a nucleoside comprising a modified sugar moiety and a deoxynucleoside,
   wherein the 5′-most central-region nucleoside is a deoxynucleoside and the 3′-most central region nucleoside is a deoxynucleoside and not more than 2 central region nucleosides comprise a modified sugar moiety; and
   wherein each internucleoside linkage within the central region that is adjacent to an adenine is a mesylphosphoramidate internucleoside linkage and the remaining internucleoside linkages of the central region are phosphorothioate internucleoside linkages.

Embodiment 67. The oligomeric compound of embodiment 66, wherein 4 nucleosides within the central region are adenine nucleosides.

Embodiment 68. The oligomeric compound of any of embodiments 62-67, wherein each 5′-region nucleoside comprises a modified sugar moiety.

Embodiment 69. Then oligomeric compound any of embodiments 62-68, wherein each 3′-region comprises a modified sugar moiety.

Embodiment 70. The oligomeric compound of any of embodiments 62-69, wherein each central-region nucleoside is a deoxynucleoside.

Embodiment 71. The oligomeric compound of any of embodiments 62-70, wherein each deoxynucleoside comprises a 2′-β-D-deoxyribosyl sugar moiety.

Embodiment 72. The oligomeric compound of any of embodiments 62-71, wherein at least one 5′-region nucleoside and/or at least one 3′-region nucleoside comprises a bicyclic sugar moiety.

Embodiment 73. The oligomeric compound of embodiment 72, wherein each bicyclic sugar moiety is selected from cEt or LNA.

Embodiment 74. The oligomeric compound of any of embodiments 62-73, wherein at least one 5′-region nucleoside and/or at least one 3′-region nucleoside comprises a 2′-substituted sugar moiety.

Embodiment 75. The oligomeric compound of any of embodiments 74 wherein the 2′-substituent of each 2′-substituted sugar moiety is selected from OCH₃, and O(CH₂)₂—OCH₃.

Embodiment 76. The oligomeric compound of any of embodiments 62-75, wherein the modified oligonucleotide comprises at least one modified nucleoside comprising a modified nucleobase.

Embodiment 77. The oligomeric compound of any of embodiments 62-76, comprising a conjugate group.

Embodiment 78. The oligomeric compound of any of embodiments 62-77, wherein each internucleoside linkage within the 5′-region is selected from a phosphodiester internucleoside linkage and a phosphorothioate internucleoside linkage.

Embodiment 79. The oligomeric compound of any of embodiments 62-78, wherein each internucleoside linkage within the 3′-region is selected from a phosphodiester internucleoside linkage and a phosphorothioate internucleoside linkage.

Embodiment 80. The oligomeric compound of any of embodiments 62-79, wherein the internucleoside linkage between the 5′-region and the central region is selected from a phosphodiester internucleoside linkage and a phosphorothioate internucleoside linkage.

Embodiment 81. The oligomeric compound of any of embodiments 62-80, wherein the internucleoside linkage between the 5′-region and the central region is a phosphodiester internucleoside linkage.

Embodiment 82. The oligomeric compound of any of embodiments 62-81, wherein the internucleoside linkage between the 3′-region and the central region is selected from a phosphodiester internucleoside linkage and a phosphorothioate internucleoside linkage.

Embodiment 83. The oligomeric compound of any of embodiments 62-82, wherein the internucleoside linkage between the 3′-region and the central region is a phosphodiester internucleoside linkage.

Embodiment 84. A method comprising administering an oligomeric compound of any of embodiments 62-83 to an animal.

Embodiment 85. The method of embodiment 58 wherein at least two doses of the oligomeric compound of any of embodiments 62-83 are administered to an animal and the oligomeric compound is administered to the animal at a dose frequency of once per 4 months, 5 months, 6 months, 7 months, 8 months, 9 months, 10 months, 11 months, or a year or more than a year.

Embodiment 86. The method of embodiment 84 or 85, wherein the oligomeric compound is dosed at an amount of 50 mg to 500 mg.

Embodiment 87. The method of any of embodiments 84-86, wherein the oligomeric compound is administered systemically.

Embodiment 88. The method of embodiment 61 or 87, wherein the oligomeric compound is administered subcutaneously.

### Certain Compounds

Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety and/or a modified nucleobase or is a stereo-non-standard nucleoside,) and/or at least one modified internucleoside linkage). Disclosed herein are oligomeric compounds comprising or consisting of modified oligonucleotides having at least one sulfonyl phosphoramidate internucleoside linkage. Such modified oligonucleotides may comprise additional modifications.

### Sulfonyl Phosphoramidate Linkages

Certain sulfonyl phosphoramidate linkages have been previously described; see, e.g., Liang, et. al., WO 2021/030778, which is hereby incorporated by reference in its entirety. In certain embodiments, compounds described herein are oligomeric compounds (including oligomeric compounds that are antisense agents or portions thereof) comprising or consisting of modified oligonucleotides consisting of linked nucleosides and having at least one sulfonyl phosphoramidate internucleoside linkage of Formula I:

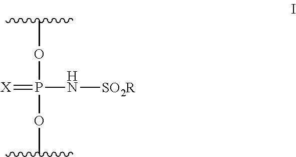

wherein:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl, substituted C₁-C₆ alkynyl, and a conjugate group. In certain embodiments, the conjugate group comprises a cell-targeting moiety. In certain embodiments, the conjugate group comprises a carbohydrate or carbohydrate cluster. In certain embodiments, the conjugate group comprises a substituted N-Acetylgalactosamine (GalNAc) moiety. In certain embodiments, the conjugate group comprises a lipid. In certain embodiments, the conjugate group comprises C₁₀-C₂₀ alkyl. In certain embodiments, the conjugate group comprises C₁₆ alkyl.

In certain embodiments, compounds described herein are oligomeric compounds (including oligomeric compounds that are antisense agents or portions thereof) comprising or consisting of oligonucleotides consisting of linked nucleosides and having at least one internucleoside linkage of Formula I, wherein:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted, and C₁-C₆ alkynyl.

In certain embodiments, compounds described herein are oligomeric compounds (including oligomeric compounds that are antisense agents or portions thereof) comprising or consisting of oligonucleotides consisting of linked nucleosides and having at least one mesyl phosphoramidate internucleoside linkage of Formula II.

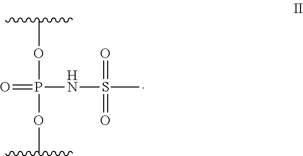 [SMILES: *OP(=O)(NS(C)(=O)=O)O*]

In certain embodiments, compounds described herein are oligomeric compounds (including oligomeric compounds that are antisense agents or portions thereof) comprising or consisting of oligonucleotides consisting of linked nucleosides and having at least one mesyl phosphoramidothioate internucleoside linkage of Formula III.

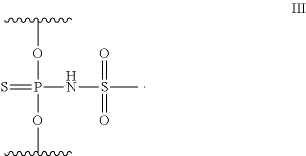 [SMILES: *OP(=S)(NS(C)(=O)=O)O*]

In certain embodiments, compounds described herein are oligomeric compounds (including oligomeric compounds that are antisense agents or portions thereof) comprising or consisting of oligonucleotides consisting of linked nucleosides and having at least one sulfonyl phosphoramidate internucleoside linkage of Formula IV.

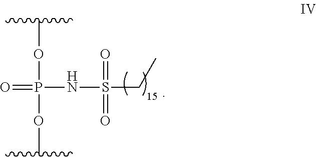

Sulfonyl phosphoramidate internucleoside linkages comprise a chiral center. Internucleoside linkages having chiral centers may be prepared as populations of modified oligonucleotides comprising stereorandom internucleoside linkages, or as populations of modified oligonucleotides comprising sulfonyl phosphoramidate linkages in particular stereochemical configurations. For example, configurations of Formula II are shown below.

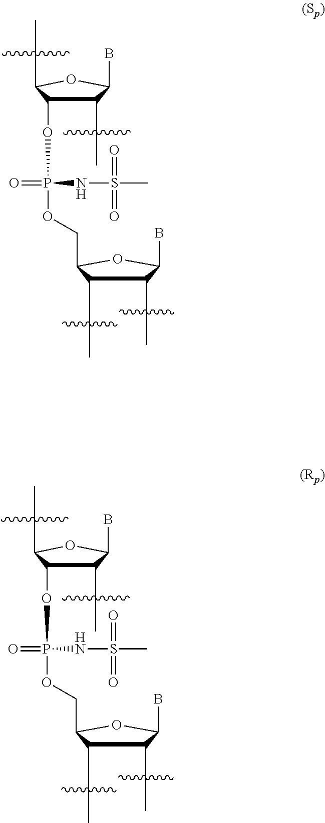

### Additional Modifications

Modified Internucleoside Linkages other than Sulfonyl Phosphoramidate Internucleoside Linkages

In certain embodiments, antisense agents, oligomeric compounds, and modified oligonucleotides comprise or consist of a modified oligonucleotide comprising one or more modified internucleoside linkages in addition to the at least one sulfonyl phosphoramidate internucleoside linkage.

The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Neutral internucleoside linkages include, without limitation, phosphotriesters, phosphonates, MMI (3′-CH₂—N(CH₃)—O-5′), amide-3 (3′—CH₂—C(═O)—N(H)—5′), amide-4 (3′—CH₂—N(H)—C(═O)—5′), formacetal (3′—O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### Chiral Internucleoside Linkages

In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACCS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

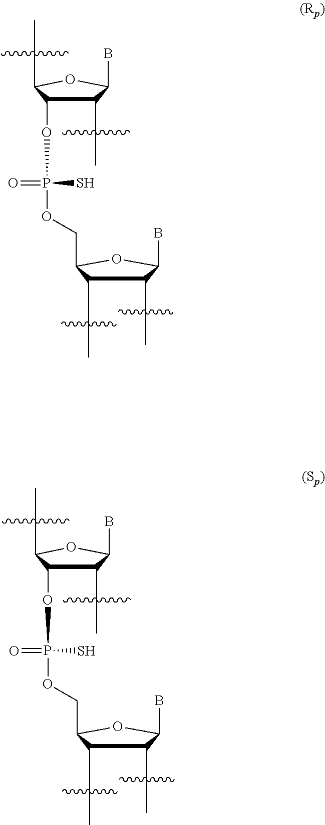

Unless otherwise indicated, chiral internucleoside linkages of modified oligonucleotides described herein can be stereorandom or in a particular stereochemical configuration.

a. Alternatives to 5′ to 3′ Internucleoside Linkages

In certain embodiments, nucleic acids can be linked 2′ to 5′ rather than the standard 3′ to 5′ linkage. Such a linkage is illustrated below.

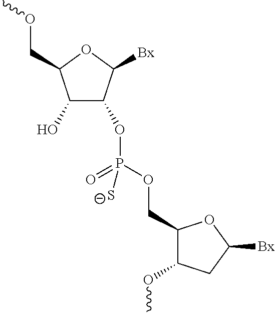

In certain embodiments, nucleosides can be linked by 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

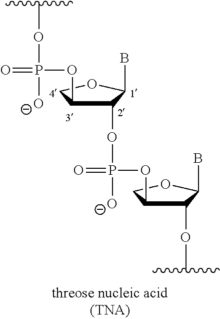

Additional modified linkages include α,β-D-CNA type linkages and related conformationally-constrained linkages, shown below. Synthesis of such molecules has been described previously (see Dupouy, et al., Angew Chem. Int. Ed Engl., 2014 45: 3623-3627; Borsting, et al. Tetrahedron, 2004, 60:10955-10966. Ostergaard, et al., ACS Chem. Biol. 2014, 9: 1975-1979; Dupouy et al., Eur. J. Org. Chem., 2008, 1285-1294; Martinez, et al, PLoS One, 2011, 6:e25510; Dupouy, et al., Eur. J. Org. Chem., 2007, 5256-5264; Boissonznet, et al., New J. Chem., 2011, 35: 1528-1533.)

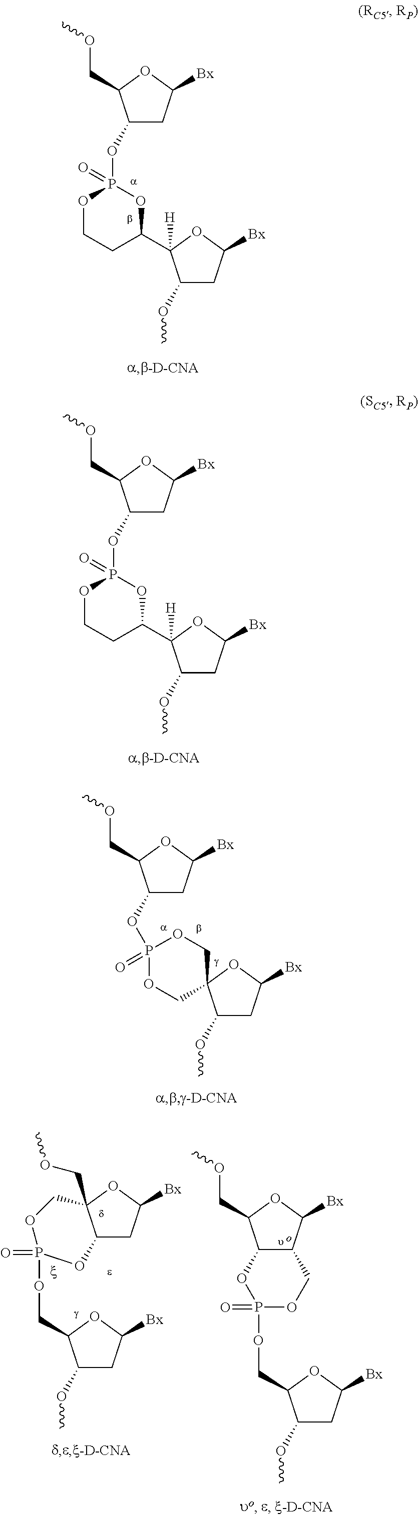

### Modified Nucleosides

In addition to the at least one sulfonyl phosphoramidate internucleoside linkage, compounds comprising or consisting of modified oligonucleotides herein may also comprise one or more modified nucleosides. Modified nucleosides include stereo-non-standard nucleosides and nucleosides comprising or a modified sugar moiety, or a modified nucleobase, or any combination thereof.

### 1. Certain Modified Sugar Moieties

In certain embodiments, modified sugar moieties are stereo-non-standard sugar moieties. In certain embodiments, sugar moieties are substituted furanosyl stereo-standard sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

a. Stereo-Non-Standard Sugar Moieties

In certain embodiments, modified sugar moieties are stereo-non-standard sugar moieties shown in Formulas V-XI below:

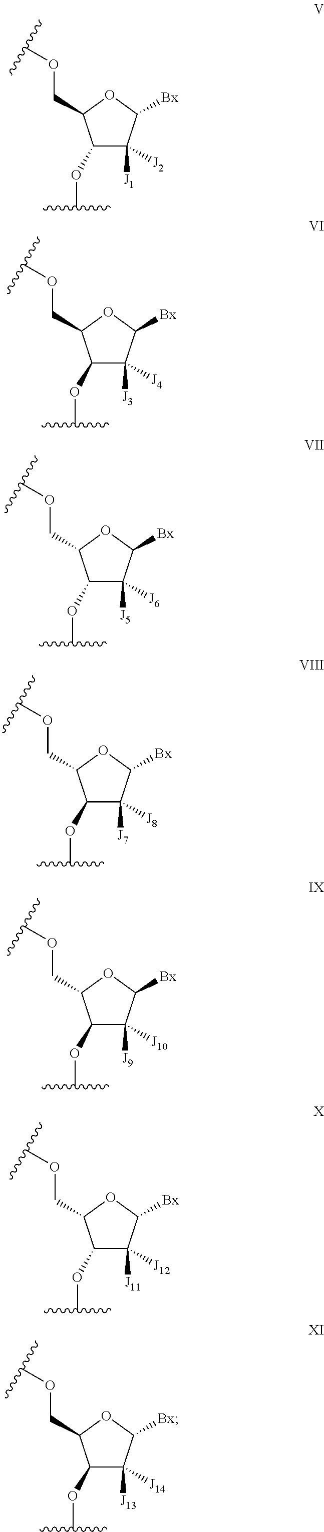

wherein one of J₁ and J₂ is H and the other of J₁ and J₂ is selected from H, OH, F, OCH₃, OCH₂CH₂OCH₃, O—C₁-C₆ alkoxy, and SCH₃;
one of J₃ and J₄ is H and the other of J₃ and J₄ is selected from H, OH, F, OCH₃, OCH₂CH₂OCH₃, O—C₁-C₆ alkoxy, and SCH₃; and wherein
one of J₅ and J₆ is H and the other of J₅ and J₆ is selected from H, OH, F, OCH₃, OCH₂CH₂OCH₃, O—C₁-C₆ alkoxy, and SCH₃; and wherein
one of J₇ and J₈ is H and the other of J₇ and J₈ is selected from H, OH, F, OCH₃, OCH₂CH₂OCH₃, O—C₁-C₆ alkoxy, and SCH₃; and wherein
one of J₉ and J₁₀ is H and the other of J₉ and J₁₀ is selected from H, OH, F, OCH₃, OCH₂CH₂OCH₃, O—C₁-C₆ alkoxy, and SCH₃; and wherein
one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein
one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and
   Bx is a is a heterocyclic base moiety.

Certain stereo-non-standard sugar moieties have been previously described in, e.g., Seth et al., WO2020/0o72991 and Seth et al., WO2019/157531, both of which are incorporated by reference herein in their entirety.

b. Substituted Stereo-Standard Sugar Moieties In certain embodiments, modified sugar moieties are substituted stereo-standard furanosyl sugar moieties comprising one or more acyclic substituent, including but not limited to substituents at the 2′, 3′, 4′, and/or 5′ positions.

In certain embodiments, the furanosyl sugar moiety is a ribosyl sugar moiety. In certain embodiments one or more acyclic substituent of substituted stereo-standard sugar moieties is branched. Examples of 2′-substituent groups suitable for substituted stereo-standard sugar moieties include but are not limited to: 2′—F, 2′—OCH₃ (“2′—OMe” or “2′—O—methyl”), and 2′—O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₘ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for substituted stereo-standard sugar moieties include but are not limited to alkoxy (e.g., methoxy), alkyl, and those described in Manoharan et al., WO 2015/106128. Examples of 5′-substituent groups suitable for substituted stereo-standard sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted stereo-standard nucleoside comprises a sugar moiety comprising a non-bridging 2′-substituent group selected from: F, NH₂, N₃, OCF₃, OCH₃, SCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₘ is, independently, H, an amino protecting group, or substituted or unsubstituted C₁-C₁₀ alkyl.

In certain embodiments, a 2′-substituted stereo-standard nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted stereo-standard nucleoside comprises a sugar moiety comprising a 2′-substituent group selected from: F, OCH₃, and OCH₂CH₂OCH₃.

In certain embodiments, the 4′ O of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

c. Bicyclic Nucleosides Certain nucleosides comprise modified sugar moieties that comprise a bridging sugar substituent that forms a second ring resulting in a bicyclic sugar moiety. In certain such embodiments, the bicyclic sugar moiety comprises a 4′ to 2′ bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′—(CH₂)₂-2′, 4′—(CH₂)₃-2′, 4′—CH₂—O—2′ (“LNA”), 4′—CH₂—S—2′, 4′—(CH₂)₂—O—2′ (“ENA”), 4′—CH(CH₃)—O-2′ (referred to as “constrained ethyl” or “cEt” when in the S configuration), 4′—CH₂—O—CH₂-2′, 4′-CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′—CH₂—N(OCH₃)—2′ and analogs thereof (see, e.g., Prakash et al., U.S. Pat. No. 8,278,425), 4′-CH₂—O—N(CH₃)—2′ (see, e.g., Allerson et al., U.S. Pat. No. 7,696,345 and Allerson et al., U.S. Pat. No. 8,124,745), 4′-CH₂—C(H)(CH₃)—2′ (see, e.g., Zhou, et al., J. Org. Chem.,2009, 74, 118-134), 4′-CH₂—C(═CH₂)—2′ and analogs thereof (see e.g., Seth et al., U.S. Pat. No. 8,278,426), 4′-C(RₐR_{b})—N(R)—O-2′, 4′—C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672), 4′-C(═O)—N(CH₃)₂-2′, 4′—C(═O)—N(R)₂-2′, 4′—C(═S)—N(R)₂-2′ and analogs thereof (see, e.g., Obika et al., WO2011052436A1, Yusuke, WO2017018360A1).

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.,; Christiansen, et al., J. Am. Chem. Soc. 1998, 120, 5458-5463; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

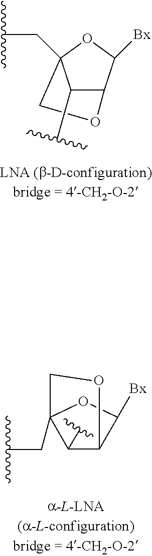

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

The term “substituted” following a position of the furanosyl ring, such as “2′-substituted” or “2′-4′-substituted”, indicates that is the only position(s) having a substituent other than those found in unmodified sugar moieties in oligonucleotides.

d. Sugar Surrogates In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, CJ. Bioorg. & Med. Chem. 2002, 10, 841-854), fluoro HNA (“F-HNA”, see e.g.Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran).

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

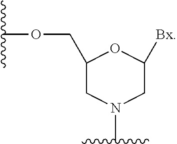

In certain embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

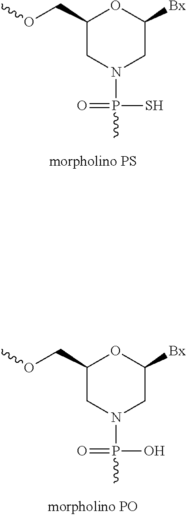

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876. In certain embodiments, acyclic sugar surrogates are selected from:

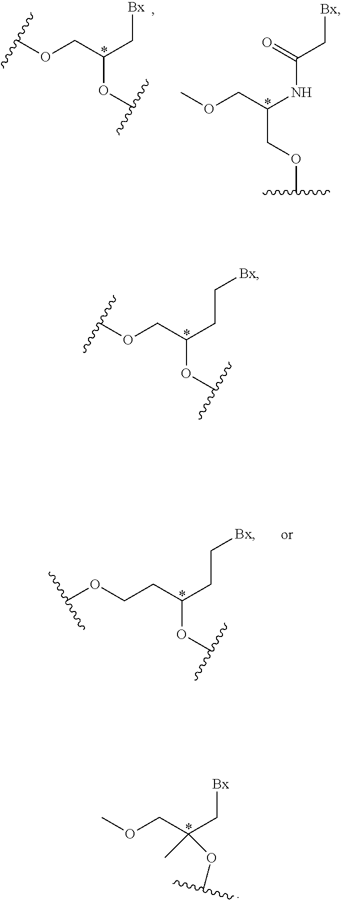

Many other bicyclic and tricyclic sugar and sugar surrogate ring systems are known in the art that can be used in modified nucleosides. Certain such ring systems are described in Hanessian, et al., J. Org. Chem., 2013, 78: 9051-9063 and include bcDNA and tcDNA. Modifications to bcDNA and tcDNA, such as 6′-fluoro, have also been described (Dogovic and Leumann, J. Org. Chem., 2014, 79: 1271-1279).

e. Conjugated Nucleosides and Terminal Groups

In certain embodiments, modified sugar moieties comprise a conjugate group and/or a terminal group. Modified sugar moieties are linked to conjugate groups through a conjugate linker. In certain embodiments, modified furanosyl sugar moieties comprise conjugate groups attached at the 2′, 3′, or 5′ positions. In certain embodiments, the 3′-most sugar moiety of the nucleoside is modified with a conjugate group or a terminal group. In certain embodiments, the 5′-most sugar moiety of the nucleoside is modified with a conjugate group or a terminal group. In certain embodiments, a sugar moiety near the 3′ end of the nucleoside is modified with a conjugate group. In certain embodiments, a sugar moiety near the 5′ end of the nucleoside is modified with a conjugate group.

Examples of terminal groups include but are not limited to conjugate groups, capping groups, phosphate group, protecting groups, modified or unmodified nucleosides, and two or more nucleosides that are independently modified or unmodified.

In certain embodiments, terminal groups at the 5′-terminus comprise a stabilized phosphate group. In certain such embodiments, the phosphorus atom of the stabilized phosphate group is attached to the 5′-terminal nucleoside through a phosphorus-carbon bond. In certain embodiments, the carbon of that phosphorus-carbon bond is in turn bound to the 5′-position of the nucleoside.

In certain embodiments, the oligonucleotide comprises a 5′-stabilized phosphate group having the following formula:

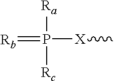

wherein:
Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
R_{b} is 0 or S;
X is substituted or unsubstituted C; and wherein X is attached to the 5′-terminal nucleoside. In certain embodiments, X is bound to an atom at the 5′-position of the 5′-terminal nucleoside. In certain such embodiments, the 5′-atom is a carbon and the bond between X and the 5′-carbon of the 5′-terminal nucleoside is a carbon-carbon single bond. In certain embodiments, it is a carbon-carbon double bond. In certain embodiments, it is a carbon-carbon triple bond. In certain embodiments, the 5′-carbon is substituted. In certain embodiments, X is substituted. In certain embodiments, X is unsubstituted.

In certain embodiments, the oligonucleotide comprises a 5′-stabilized phosphate group having the following formula:

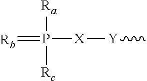

wherein:
Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
R_{b} is 0 or S;
X is substituted or unsubstituted C;
Y is selected from C, S, and N. In certain embodiments, Y is substituted or unsubstituted C. The bond between X and Y may be a single-, double-, or triple-bond.

Certain 5′-stabilized phosphate groups have been previously described; see, e.g., Prakash et al., WO2011/139699 and Prakash et al., WO2011/139702, hereby incorporated by reference herein in their entirety.

In certain embodiments, the stabilized phosphate group is 5′-vinyl phosphonate or 5′-cyclopropyl phosphonate.

In certain embodiments, a terminal group at the 5′-terminus is a 5′-mesyl phosphoramidate, having formula XII:

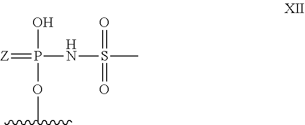

wherein Z is O or S.

In certain embodiments, a terminal group at the 5′-terminus is a 5′-mesyl phosphoramidate, having formula XIII:

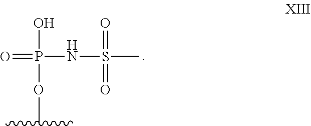 [SMILES: *OP(=O)(O)NS(C)(=O)=O]

### 2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906;; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to a target nucleic acid comprising one or more modified nucleobases. In certain embodiments, the modified nucleobase is 5-methylcytosine. In certain embodiments, each cytosine is a 5-methylcytosine.

### Certain Conjugate Groups and Conjugate Moieties

In certain embodiments, oligomeric compounds comprise one or more conjugate moieties or conjugate groups. In certain embodiments, conjugate groups modify one or more properties of the molecule, including but not limited to pharmacodynamics, pharmacokinetics, stability, binding, absorption, tissue distribution, cellular distribution, cellular uptake, charge and clearance. In certain embodiments, conjugate moieties impart a new property on the molecule, e.g., fluorophores or reporter groups that enable detection of the molecule.

Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937),_a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

a. Conjugate Moieties Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

b. Conjugate Linkers

In certain embodiments, conjugate groups comprise a conjugate linker that attaches a conjugate moiety to the remainder of the modified oligonucleotide. In certain embodiments, a conjugate linker is a single chemical bond (i.e. conjugate moiety is attached to the remainder of the modified oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group or conjugate moiety to be cleaved from the remainder of the oligonucleotide. For example, in certain circumstances oligomeric compounds (including oligomeric compounds that are antisense agents or portions thereof) or modified oligonucleotides comprising a particular conjugate moiety are better taken up by a particular cell type, but once the compound has been taken up, it is desirable that the conjugate group be cleaved to release an unconjugated oligonucleotide. Thus, certain conjugate moieties may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

c. Certain Cell-Targeting Conjugate Moieties

In certain embodiments, a conjugate group comprises a cell-targeting conjugate moiety. In certain embodiments, a conjugate group has the general formula:

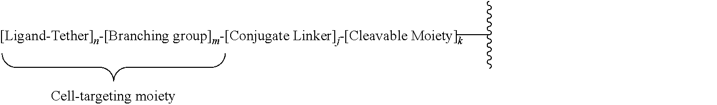

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycolyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds (including oligomeric compounds that are antisense agents or portions thereof) or modified oligonucleotides described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### I. Certain Motifs

In certain embodiments, antisense agents, oligomeric compounds, and modified oligonucleotides described herein comprise or consist of oligonucleotides. Modified oligonucleotides can be described by their motif, e.g. a pattern of unmodified and/or modified sugar moieties, nucleobases, and/or internucleoside linkages. In certain embodiments, modified oligonucleotides comprise one or more stereo-non-standard nucleosides. In certain embodiments, modified oligonucleotides comprise one or more stereo-standard nucleosides. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

A. Certain Sugar Motifs In certain embodiments, antisense agents, oligomeric compounds, and modified oligonucleotides described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include without limitation any of the sugar modifications discussed herein.

In certain embodiments, a modified oligonucleotide comprises or consists of a gapmer. The sugar motif of a gapmer defines the regions of the gapmer: 5′-region, central region (gap), and 3′-region. The central region is linked directly to the 5′-region and to the 3′-region with no nucleosides intervening. The central region is a deoxy region. The nucleoside at the first position (position 1) from the 5′-end of the central region and the nucleoside at the last position of the central region are adjacent to the 5′-region and 3′-region, respectively, and each comprise a sugar moiety independently selected from a 2′-deoxyfuranosyl sugar moiety or a sugar surrogate. In certain embodiments, the nucleoside at position 1 of the central region and the nucleoside at the last position of the central region are DNA nucleosides, selected from stereo-standard DNA nucleosides or stereo-non-standard DNA nucleosidesV-XI. Unlike the nucleosides at the first and last positions of the central region, the nucleosides at the other positions within the central region may comprise a 2′-substituted furanosyl sugar moiety or a substituted stereo-non-standard sugar moiety or a bicyclic sugar moiety. In certain embodiments, each nucleoside within the central region supports RNase H cleavage. In certain embodiments, a plurality of nucleosides within the central region support RNase H cleavage.

Herein, the lengths (number of nucleosides) of the three regions of a gapmer may be provided using the notation [# of nucleosides in the 5′-region]-[# of nucleosides in the central region]-[# of nucleosides in the 3′-region]. Thus, a 3-10-3 gapmer consists of 3 linked nucleosides in each of the 3′ and 5′ regions and 10 linked nucleosides in the central region. Where such nomenclature is followed by a specific modification, that modification is the modification of each sugar moiety of each 5′ and 3′-region and the central region nucleosides comprise stereo-standard DNA sugar moieties. Thus, a 5-10-5 MOE gapmer consists of 5 linked nucleosides each comprising 2′-MOE-stereo-standard sugar moieties in the 5′-region, 10 linked nucleosides each comprising a stereo-standard DNA sugar moiety in the central region, and 5 linked nucleosides each comprising 2′-MOE-stereo-standard sugar moieties in the 3′-region. A 5-10-5 MOE gapmer having a substituted stereo-non-standard nucleoside at position 2 of the gap has a gap of 10 nucleosides wherein the 2^{nd} a nucleoside of the gap is a substituted stereo-non-standard nucleoside rather than the stereo-standard DNA nucleoside. Such oligonucleotide may also be described as a 5-1-1-8-5 MOE/substituted stereo-non-standard/MOE gapmer. A 3-10-3 cEt gapmer consists of 3 linked nucleosides each comprising a cEt in the 5′-region, 10 linked nucleosides each comprising a stereo-standard DNA sugar moiety in the central region, and 3 linked nucleosides each comprising a cEt in the 3′-region. A 3-10-3 cEt gapmer having a substituted stereo-non-standard nucleoside at position 2 of the gap has a gap of 10 nucleoside wherein the 2^{nd} nucleoside of the gap is a substituted stereo-non-standard nucleoside rather than the stereo-standard DNA nucleoside. Such oligonucleotide may also be described as a 3-1-1-8-3 cEt/substituted stereo-non-standard/cEt gapmer.

The sugar motif of a 3-10-3 cEt gapmer may also be denoted by the notation kkk-d(10)-kkk, wherein each “k” represents a cEt and each “d” represents a 2′-β-D-deoxyribosyl sugar moiety. This sugar motif is independent of the nucleobase sequence, the internucleoside linkage motif, and any nucleobase modifications. A 5-10-5 MOE gapmer may be denoted by the notation eeeee-d(10)-eeeee or e(5)-d(10)-e(5), wherein each “e” represents a 2′-MOE-β-D-ribofuranosyl sugar moiety, and each “d” represents a 2′-β-D-deoxyribosyl sugar moiety.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

### B. Certain Nucleobase Motifs

In certain embodiments antisense agents, oligomeric compounds, and modified oligonucleotides described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

C. Certain Internucleoside Linkage Motifs In certain embodiments, antisense agents, oligomeric compounds, and modified oligonucleotides described herein comprise or consist of oligonucleotides. In certain embodiments, oligonucleotides comprise modified and/or unmodified internucleoside linkages arranged along the oligonucleotide or region thereof in a defined pattern or motif.

In certain embodiments, each internucleoside linkage is selected from a sulfonyl phosphoramidate internucleoside linkage, a phosphorothioate internucleoside linkage, and a phosphodiester internucleoside linkage. In certain embodiments, each internucleoside linkage of a modified oligonucleotide is selected from a sulfonyl phosphoramidate linkage and a phosphorothioate internucleoside linkage. In certain embodiments, each internucleoside linkage is selected from sulfonyl phosphoramidate internucleoside linkage and a phosphodiester internucleoside linkage. In certain embodiments, each sulfonyl phosphoramidate internucleoside linkage has Formula II.

III. Certain Modified Oligonucleotides In certain embodiments, antisense agents, oligomeric compounds, and modified oligonucleotides described herein comprise or consist of modified oligonucleotides. In certain embodiments, the above modifications (sugar, nucleobase, internucleoside linkage) are incorporated into a modified oligonucleotide. Modified oligonucleotides can be described by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. In certain embodiments, motifs are described relative to another motif. For example, a modified oligonucleotide may described as having a region defined by a sugar motif (e.g., a deoxy region) and the internucleoside linkage motife may recite particular internucleoside linkages withing that sugar-motif defined region. In sush instances, the term “within” refers to each internucleoside linkage between nucleosides, both of which are within the sugar-motif defined region.

In certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with a RNA-like nucleoside, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### Compositions and Methods for Formulating Pharmaceutical Compositions

Antisense agents, oligomeric compounds, and modified oligonucleotides described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds (including oligomeric compounds that are antisense agents or portions thereof) or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds (including oligomeric compounds that are antisense agents or portions thereof) provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

In certain embodiments, oligomeric compounds (including oligomeric compounds that are antisense agents or portions thereof) described herein comprise or consist of modified oligonucleotides. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Nucleosides that are sufficiently “DNA-like” to elicit RNase H activity are referred to as DNA mimics herein. Further, in certain embodiments, one or more non-DNA-like nucleoside in in the RNA:DNA duplex is tolerated.

In certain antisense activities, hybridization of an antisense agent, oligomeric compound, or modified oligonucleotide described herein to a target nucleic acid results in modulation of the splicing of a target pre-mRNA. For example, in certain embodiments, hybridization of a compound described herein will increase exclusion of an exon. For example, in certain embodiments, hybridization of a compound described herein will increase inclusion of an exon.

In certain antisense activities, antisense agents described herein or a portion of the antisense agent is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi agents. RNAi agents may be double-stranded (siRNA) or single-stranded (ssRNA).

In certain antisense activities, antisense agents, oligomeric compounds, or modified oligonucleotides described herein result in a CRISPR system cleaving a target DNA. In certain antisense activities, compounds described herein result in a CRISPR system editing a target DNA.

In certain antisense activities, hybridization of an antisense agent, oligomeric compound, or modified oligonucleotide described herein to a target nucleic acid results in disruption of secondary structural elements, such as stem-loops and hairpins. For example, in certain embodiments, hybridization of a compound described herein to a stem-loop that is part of a translation suppression element leads to an increase in protein expression.

In certain antisense activities, hybridization of an antisense agent, oligomeric compound, or modified oligonucleotide described herein to a target nucleic acid leads to no-go decay mediated mRNA degradation.

In certain antisense activities, hybridization of an antisense agent, oligomeric compound, or modified oligonucleotide described herein to a target nucleic acid leads to activation of nonsense-mediated decay mRNA degradation.

In certain embodiments, antisense agents, oligomeric compounds, or modified oligonucleotides described herein are artificial mRNA compounds, the nucleobase sequence of which encodes for a protein.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Certain RNAi Agents

In certain embodiments, oligomeric compounds described herein having one or more stinternucleoside linkages Formula I are RNAi agents. In certain embodiments, internucleoside linkages having Formula I can replace one or more phosphorothioate or phosphodiester intemucleoside linkages in any RNAi motif. Certain RNAi motifs are described in, e.g., Freier, et al., WO2020/160163, incorporated by reference herein in its entirety; as well as, e.g., Rajeev, et al., WO2013/075035; Maier, et al., WO2016/028649; Theile, et al., WO2018/098328; Nair, et al., WO2019/217459; each of which is incorporated by reference herein.

### Target Nucleic Acids, Target Regions and Nucleotide Sequences

In certain embodiments, antisense agents, oligomeric compounds, or modified oligonucleotides described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. In certain embodiments, the target nucleic acid is a microRNA. In certain embodiments, the target region is in the 5′ UTR of a gene. In certain embodiments, the target region is within a translation suppression element region of a target nucleic acid.

### Certain Modified Sulfonyl Phosphormidate Containing Compounds and Properties

In certain embodiments, antisense agents, oligomeric compounds, and modified oligonucleotides comprising at least one sulfonyl phosphoramidate linkage have desirable properties. In certain embodiments, antisense agents, oligomeric compounds, and modified oligonucleotides comprising at least one sulfonyl phosphoramidate linkage have improved properties relative to compounds that are otherwise the same, but lack the sulfonyl phosphoramidate internucleoside linkages (e.g., have phosphorothioate or phosphodiester intemucleoside linkages at those positions).

For example, in certain embodiments, antisense agents, oligomeric compounds, and modified oligonucleotides described herein having one or more mesyl phosphoramidate internucleoside linkages have reduced hepatotoxicity. In certain embodiments, antisense agents, oligomeric compounds, and modified oligonucleotides described herein having one or more mesyl phosphoramidate internucleoside linkages have increased stability in the presence of nucleases. In certain embodiments, antisense agents, oligomeric compounds, and modified oligonucleotides described herein having one or more mesyl phosphoramidate internucleoside linkages have improved duration of action. In certain embodiments, antisense agents, oligomeric compounds, and modified oligonucleotides described herein having one or more mesyl phosphoramidate internucleoside linkages have reduced hepatotoxicity, increased stability in the presence of nucleases, and improved duration of action. In certain embodiments, antisense agents, oligomeric compounds, and modified oligonucleotides described herein having one or more mesyl phosphoramidate internucleoside linkages have reduced interactions with certain proteins. In certain embodiments, antisense agents, oligomeric compounds, and modified oligonucleotides described herein having one or more mesyl phosphoramidate internucleoside linkages have increased interactions with certain proteins.

Sulfonyl Phosphoramidate Linkages within Deoxy Regions

In certain embodiments, antisense agents, oligomeric compounds, and modified oligonucleotides have a sugar motif comprising a deoxy region and comprise one sulfonyl phosphoramidate internucleoside linkage within that deoxy region. In certain embodiments, antisense agents, oligomeric compounds, and modified oligonucleotides have a sugar motif comprising a deoxy region and comprise a block of two mesyl phosphoramidate internucleoside linkages in a row within that deoxy region. Certain such modified oligonucleotides comprising blocks of mesyl phosphoramidate internucleoside linkages have long duration of action and are amenable to infrequent dosing to a subject described below.

In certain embodiments, antisense agents, oligomeric compounds, and modified oligonucleotides have a sugar motif comprising a deoxy region wherein each internucleoside linkages within the deoxy region that links to the 3′ side of a purine nucleoside (e.g., each 3′-adjacent linkage) is a mesyl phosphoramidate internucleoside linkage. In certain embodiments, antisense agents, oligomeric compounds, and modified oligonucleotides have a sugar motif comprising a deoxy region wherein each internucleoside linkages within the deoxy region that links to the 5′ side of a purine nucleoside (e.g., each 5′-adjacent linkage) is a mesyl phosphoramidate internucleoside linkage. In certain embodiments, antisense agents, oligomeric compounds, and modified oligonucleotides have a sugar motif comprising a deoxy region wherein each internucleoside linkages within the deoxy region that links to the 3′ side and each internucleoside linkages within the deoxy region that links to the 5′ side of a purine nucleoside is a mesyl phosphoramidate internucleoside linkage. In certain of the above embodiments, the internucleoside linkages within the deoxy region (that do not link to a purine as described above) are selected from phosphorothioate internucleoside linkages and phosphodiester internucleoside linkages. Certain such modified oligonucleotides comprising a region of alternating mesyl phosphoramidate internucleoside linkages have long duration of action and are amenable to infrequent dosing to a subject described below. In certain embodiments, each deoxynucleoside comprising a purine nucleobase, e.g., an adenine or a guanine, comprises a 3′-adjacent and/or a 5′-adjacent mesyl phosphoramidate internucleoside linkage. In certain embodiments, each internucleoside linkage adjacent to a deoxynucleoside comprising a purine nucleobase is a mesyl phosphoramidate internucleoside linkage. In certain embodiments, each internucleoside linkage in the central region of a modified oligonucleotide adjacent to a deoxynucleoside comprising a purine nucleobase is a mesyl phosphoramidate internucleoside linkage. In certain embodiments, each mesyl phosphoramidate internucleoside linkage in an oligomeric compound is adjacent to a deoxynucleoside comprising a purine nucleobase. In certain embodiments, each mesyl phosphoramidate internucleoside linkage in a modified oligonucleotide is in the central region of the modified oligonucleotide and adjacent to a deoxynucleoside comprising a purine nucleobase.

In each of the above embodiments, the deoxy region may be flanked on the 5′-end by a 5′-region and on the 3′-end by a 3′-region, wherein the 5′-region and the 3′-region are not deoxy regions. In such embodiment, the deoxy region may be referred to as a central region. In certain such embodiments, the internucleoside linkages between the 5′-region and the central region and between the central region and the 3′-region are selected from mesyl phosphoramidate internucleoside linkages, phosphorothioate internucleoside linkages, and phosphodiester internucleoside linkages; or from phosphorothioate internucleoside linkages and phosphodiester internucleoside linkages. In certain of the above embodiments, the modified oligonculeotides are gapmers.

In certain embodiments, such compounds have extended duration of action, while retaining activity and without creating toxicity, such as hepatotoxicity.

### IV. Certain Dosing Regimens

In certain embodiments, described herein are methods of administering to a subject a therapeutically effective amount of an oligomeric compound comprising a modified oligonucleotide as described above. In certain embodiments, methods comprise administering the therapeutically effective amount infrequently (e.g., permitting a longer dosing interval than an analogous compound having a phosphodiester or phosphorothioate internucleoside linkage in place of each mesyl phosphoramidate linkage). As described herein, certain oligomeric compounds comprising at least one mesyl phosphormidate internucleoside linkage have long duration of action. In experiments conducted in mice, certain such compounds demonstrated duration of action consistent with dosing in humans at low frequency. In certain embodments, compounds described herein are dosed in a subject, including a human, once about every four months. In certain embodiments, methods comprise administering the therapeutically effective amount once about every five months. In certain embodiments, methods comprise administering the therapeutically effective amount once about every six months. In certain embodiments, methods comprise administering the therapeutically effective amount once about every seven month. In certain embodiments, methods comprise administering the therapeutically effective amount once about every eight months. In certain embodiments, methods comprise administering the therapeutically effective amount once about every nine months. In certain embodiments, methods comprise administering the therapeutically effective amount once about every ten months. In certain embodiments, methods comprise administering the therapeutically effective amount once about every eleven months. In certain embodiments, methods comprise administering the therapeutically effective amount once about every twelve months or more than twelve months. As is understood in the field, dosing frequencies do not require exact timing between doses. For example, dosing “every twelve months” does not require each dose be administered on the precise anniversary date of the previous dose. Rather, doses are typically administered within +/−5% to 10% of the time prescribed.

In certain of the dosing embodiments described above, the dose amount may be selected from 0.1 mg/kg to 10 mg/kg of oligomeric compound comprising at least one mesyl phosphormidate internucleoside linkage. In certain embodiments, the oligomeric compound comprising at least one mesyl phosphormidate internucleoside linkage is administered at a total dose of 50 mg to 500 mg. In certain such embodiments, the oligomeric compounds comprising at least one mesyl phosphormidate internucleoside linkage are administered systemically. In certain such embodiments, the oligomeric compound is administered subcutaneously.

### Certain Compounds/Conventions

Certain compounds described herein (e.g., antisense agents, oligomeric compounds, and modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³³SS, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples are intended to illustrate certain aspects of the invention and are not intended to limit the invention in any way.

### Example 1: Synthesis of Oligomeric Compounds Comprising Modified Oligonucleotides with Mesyl Phosphoramidate Internucleoside Linkages

Oligomeric compounds comprising modified oligonucleotides comprising mesyl phosphoramidate internucleoside linkages were synthesized and tested. As shown in Table 1 and Table 2, each of the modified oligonucleotides has the same nucleobase sequence, AGCACTTTATTGAGTT (SEQ ID NO: 2), which is 100% complementary to mouse FXII genomic sequence represented by the complement to GENBANK Accession No. NC_000079.6 truncated from nucleobases 55415001 to 55430000 (SEQ ID NO: 1), at positions 12009 to 12024.

The modified oligonucleotides in the Table 1 below are all conjugated to a THA-GalNAc conjugate (designated as “THA-GalNAc”) at the 5′ end of the modified oligonucleotide. THA-GalNAc is represented by the structure below, wherein the phosphate group is attached to the 5′-oxygen atom of the 5′-nucleoside:

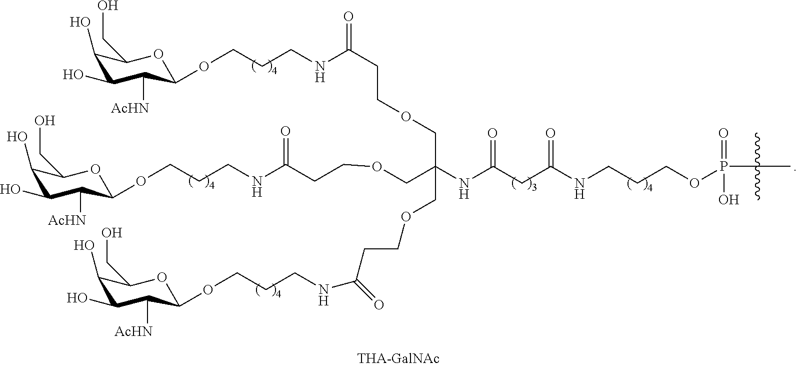

TABLE 1
Design of oligomeric compounds with mesyl phosphoramidate internucleoside linkages

| Compound | | SEQ |
|---|---|---|
| No. | Chemical Notation (5′ to 3′) | ID NO |
| 1447171 | THA-GalNAc-AₖₛGₖₛ^{m}CₖₛA_{ds}^{m}C_{ds}T_{ds}T_{ds}T_{ds}A_{ds}T_{ds}T_{ds}G_{ds}A_{ds}GₖₛTₖₛTₖ | 5 |
| 1563866 | THA-GalNAc-AₖₛGₖₛ^{m}CₖₛA_{dz}^{m}C_{ds}T_{ds}T_{ds}T_{ds}A_{ds}T_{ds}T_{ds}G_{ds}A_{ds}GₖₛTₖₛTₖ | 8 |
| 1563867 | THA-GalNAc-AₖₛGₖₛ^{m}CₖₛA_{ds}^{m}C_{ds}T_{ds}T_{ds}T_{ds}A_{ds}T_{ds}T_{ds}G_{dz}A_{ds}GₖₛTₖₛTₖ | 9 |
| 1563868 | THA-GalNAc-AₖₛGₖₛ^{m}CₖₛA_{ds}^{m}C_{ds}T_{ds}T_{ds}T_{ds}A_{dz}T_{ds}T_{ds}G_{ds}A_{ds}GₖₛTₖₛTₖ | 10 |
| 1563869 | THA-GalNAc-AₖₛGₖₛ^{m}CₖₛA_{ds}^{m}C_{ds}T_{ds}T_{ds}T_{dz}A_{ds}T_{ds}T_{ds}G_{ds}A_{ds}GₖₛTₖₛTₖ | 11 |
| 1563870 | THA-GalNAc-AₖₛGₖₛ^{m}CₖₛA_{ds}^{m}C_{ds}T_{ds}T_{ds}T_{dz}A_{dz}T_{ds}T_{ds}G_{ds}A_{ds}GₖₛTₖₛTₖ | 12 |
| 1563871 | THA-GalNAc-AₖₛGₖₛ^{m}CₖₛA_{dz}^{m}C_{ds}T_{ds}T_{ds}T_{dz}A_{dz}T_{ds}T_{ds}G_{dz}A_{ds}GₖₛTₖₛTₖ | 13 |
| 1563872 | THA-GalNAc-AₖₛGₖₛ^{m}CₖₛA_{dz}^{m}C_{ds}T_{ds}T_{ds}T_{dz}A_{dz}T_{ds}T_{dz}G_{dz}A_{ds}GₖₛTₖₛTₖ | 14 |

A subscript “k” represents a cEt modified sugar moiety, a subscript “d” represents a stereo-standard DNA nucleoside, a subscript “s” indicates a phosphorothioate internucleoside linkage, and a subscript “z” represents a mesyl phosphoramidate internucleoside linkage. A superscript “m” before a C represents a 5-methylcytosine nucleobase. THA-GalNAc represents

### A 5′-Galnac Moiety.

### Example 2: Design of an RNAi Compound with Antisense RNAi Oligonucleotide Complementary to Mouse FXII Nucleic Acid

An RNAi compound comprising an antisense RNAi oligonucleotide complementary to a mouse FXII nucleic acid, and a sense RNAi oligonucleotide complementary to the antisense RNAi oligonucleotides were designed as follows.

The antisense strand described in the table below has the sequence UAAAGCACUUUAUUGAGUUUCUG (SEQ ID NO: 3). Aside from a single mismatch at position 1 on the 5′-end, the antisense strand is 100% complementary to the complement of GenBank Accession No. NC_000079.6, truncated from nucleosides 55415001 to 55430000, (SEQ ID NO: 1) from nucleosides 12005 to 12026.

TABLE 2
Design of antisense RNAi oligonucleotide targeted to mouse FXII

| Compound | | SEQ ID |
|---|---|---|
| No. | Chemistry Notation (5′ to 3′) | NO. |
| 1523579 | U_{ys}A_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 6 |

In the table above, a subscript “f” represents a 2′-F modified sugar moiety, a subscript “y” represents a 2′-OMe modified sugar moiety, a subscript “s” represents a phosphorothioate internucleoside linkage, and a subscript “o” represents a phosphodiester internucleoside linkage.

The sense oligonucleotide is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides). Each sense strand further contains a GalNAc moiety conjugated to the 3′-oxygen as shown below:

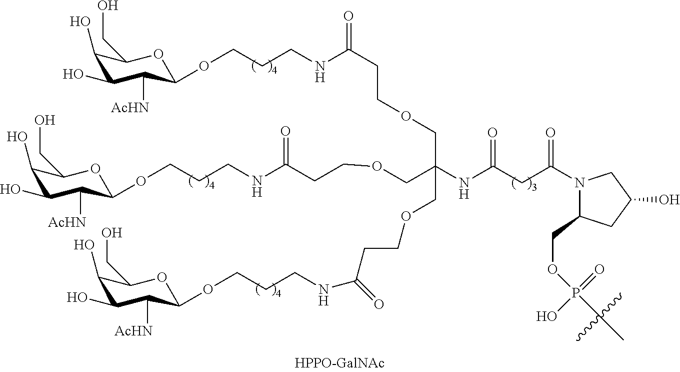

TABLE 3
Design of sense strand modified oligonucleotides targeted to mouse FXII

| Compound | | SEQ ID |
|---|---|---|
| No. | Chemistry Notation (5′ to 3′) | NO. |
| 1523578 | G_{ys}A_{ys}A_{yo}A_{yo}C_{yo}U_{yo}C_{fo}A_{yo}A_{fo}U_{fo}A_{fo}A_{yo}A_{yo}G_{yo}U_{yo}G_{yo}C_{yo}U_{yo}U_{yo}U_{yo}A_{y}-HPPO-GalNAc | 7 |

In the table above, a subscript “f” represents a 2′-F modified sugar moiety, a subscript “y” represents a 2′-OMe modified sugar moiety, a subscript “s” represents a phosphorothioate internucleoside linkage, and a subscript “o” represents a phosphodiester internucleoside linkage.

TABLE 4
Design of siRNA targeted to mouse FXII

| Duplex | Antisense Strand | Sense Strand |
|---|---|---|
| Compound No. | Compound No. | Compound No. |
| 1523582 | 1523579 | 1523578 |

### Example 3: Long Term In Vivo Activity of Modified Oligonucleotides with Mesyl Phosphoramidate Internucleoside Linkages and RNAi Agents Complementary to Mouse FXII RNA in Wild-Type Mice, Single Dose

Wildtype C₅₇BL/6 mice were treated with modified oligonucleotides and RNAi agents selected from those described above.

### STUDY 1

### Treatment

Wildtype C₅₇BL/6 mice (Jackson Laboratory) were divided into groups of 4 male mice each for treatment. Mice received a single subcutaneous injection of one of the oligomeric compounds described above at dose of 0.1645 μmol/kg. One group of 4 male mice were left untreated and are labeled “Naïve” in the table below. These “Naïve” mice served as the control group to which oligonucleotide-treated groups were compared.

### Protein Analysis

Blood plasma samples were collected at various timepoints which are indicated in the column labeled “Timepoint (Days)” in the table below. Mouse FXII protein levels in plasma were determined using a Molecular Innovations FXII ELISA kit (Cat #MFXIIKT-TOT). Reduction of F12 protein was calculated in the table below as percent FXII protein change relative to the amount of FXII protein in the same animals at the start of treatment on Day 0 normalized to naïve untreated animals (% Baseline).
“N/A” refers to samples that were not available.

TABLE 7
Mouse FXII protein levels in wildtype C57BL/6 mice

| Compound | Timepoint | FXII protein (% |
|---|---|---|
| No. | (Days) | Baseline) in plasma |
| Naïve | 0 | 100 |
| | 7 | 100 |
| | 14 | 100 |
| | 21 | 101 |
| | 28 | 103‡ |
| | 35 | 101 |
| | 42 | 101 |
| 1447171 | 0 | 100 |
| | 7 | 10‡ |
| | 14 | 15 |
| | 21 | 21 |
| | 28 | 60 |
| | 35 | 60 |
| | 42 | 77‡ |
| 1523582 | 0 | 100 |
| | 7 | 17‡ |
| | 14 | 11‡ |
| | 21 | 3 |
| | 28 | 6 |
| | 35 | 8 |
| | 42 | 9 |
| 1563866 | 0 | 100 |
| | 7 | N/A |
| | 14 | 17 |
| | 21 | 20 |
| | 28 | 63 |
| | 35 | 45 |
| | 42 | 60‡ |
| 1563867 | 0 | 100 |
| | 7 | 20‡ |
| | 14 | 29 |
| | 21 | 19 |
| | 28 | 58 |
| | 35 | 53 |
| | 42 | 64 |
| 1563868 | 0 | 100 |
| | 7 | N/A |
| | 14 | 22 |
| | 21 | 17 |
| | 28 | 45 |
| | 35 | 51 |
| | 42 | 51‡ |
| 1563869 | 0 | 100 |
| | 7 | 15‡ |
| | 14 | 28 |
| | 21 | 22 |
| | 28 | 42 |
| | 35 | 53 |
| | 42 | 38‡ |
| 1563870 | 0 | 100 |
| | 7 | 15 |
| | 14 | 20 |
| | 21 | 12 |
| | 28 | 26 |
| | 35 | 29 |
| | 42 | 36 |
| 1563871 | 0 | 100 |
| | 7 | 16‡ |
| | 14 | 18‡ |
| | 21 | 12 |
| | 28 | 34 |
| | 35 | 27 |
| | 42 | 54‡ |
| 1563872 | 0 | 100 |
| | 7 | 16 |
| | 14 | 15‡ |
| | 21 | 10 |
| | 28 | 15 |
| | 35 | 11 |
| | 42 | 17‡ |

‡indicates that fewer than 4 samples were available

What is claimed:

1. An oligomeric compound comprising a modified oligonucleotide consisting of 12-30 linked nucleosides,
wherein the modified oligonucleotide comprises:
a 5′-region consisting of 2-5 linked 5′-region nucleosides, wherein the 3′-most 5′-region nucleoside comprises a modified sugar moiety;
a 3′-region consisting of 2-5 linked 3′-region nucleosides, wherein the 5′-most 3′-region nucleoside comprises a modified sugar moiety;
a central region between the 5′-region and 3′-region consisting of 8-12 linked central-region nucleosides, wherein
each central region nucleoside is independently selected from a nucleoside comprising a modified sugar moiety and a deoxynucleoside, and at least one central region nucleoside comprises a purine nucleoside;
wherein the 5′-most central-region nucleoside is a deoxynucleoside and the 3′-most central region nucleoside is a deoxynucleoside and not more than 2 central region nucleosides comprise a modified sugar moiety; and
wherein at least one internucleoside linkage adjacent to a purine nucleoside within the central region is a methanesulfonyl phosphoramidate linkage.

2. The oligomeric compound of claim 1, wherein each internucleoside linkage within the central region that is immediately 5′ of a purine nucleoside is a methanesulfonylphosphoramidate internucleoside linkage.

3. The oligomeric compound of claim 1 or 2, wherein each internucleoside linkage within the central region that is immediately 3′ of a purine nucleoside is a methane sulfonylphosphoramidate internucleoside linkage.

4. The oligomeric compound of any one of claim 1-3, wherein each internucleoside linkage that is not immediately 3′ or 5′ of a purine nucleoside is selected from a phosphorothioate internucleoside linkage and a phosphodiester internucleoside linkage.

5. The oligomeric compound of any one of claim 1-4, wherein each internucleoside linkage that is not immediately 3′ or 5′ of a purine nucleoside is a phosphorothioate internucleoside linkage.

6. The oligomeric compound of any of claims 1-4, wherein 5 nucleosides within the central region are purine nucleosides.

7. The oligomeric compound of any of claims 1-4, wherein 4 nucleosides within the central region are purine nucleosides.

8. The oligomeric compound of any of claims 1-7, wherein each internucleoside linkage within the central region that is immediately 5′ of an adenine nucleoside is a methanesulfonylphosphoramidate internucleoside linkage.

9. The oligomeric compound of any of claims 1-8, wherein each internucleoside linkage within the central region that is immediately 3′ of an adenine nucleoside is a methanesulfonylphosphoramidate internucleoside linkage.

10. The oligomeric compound of claim 8 or 9, wherein each internucleoside linkage that is not immediately 3′ or 5′ of an adenine nucleoside is a phosphorothioate internucleoside linkage or a phosphodiester internucleoside linkage.

11. The oligomeric compound of any one of claims 8-10, wherein each internucleoside linkage that is not immediately 3′ or 5′ of an adenine nucleoside is a phosphorothioate internucleoside linkage.

12. The oligomeric compound of any of claims 8-11, wherein 4 nucleosides within the central region are adenine nucleosides.

13. The oligomeric compound of any of claims 8-11, wherein two nucleosides within the central region are adenine nucleosides.

14. The oligomeric compound of any of claims 1-13, wherein each internucleoside linkage within the central region that is immediately 5′ of a guanine nucleoside is a methanesulfonylphosphoramidate internucleoside linkage.

15. The oligomeric compound of any of claims 1-14, wherein each internucleoside linkage within the central region that is immediately 3′ of a guanine nucleoside is a methanesulfonylphosphoramidate internucleoside linkage.

16. The oligomeric compound of claim 14 or 15, wherein each internucleoside linkage that is not immediately 3′ or 5′ of a guanine nucleoside is a phosphorothioate internucleoside linkage or a phosphodiester internucleoside linkage.

17. The oligomeric compound of claim 14 or 15, wherein each internucleoside linkage that is not immediately 3′ or 5′ of a guanine nucleoside is a phosphorothioate internucleoside linkage.

18. The oligomeric compound of any of claims 1-17, wherein each methanesulfonylphosphoramidate internucleoside linkage is immediately 5′ or 3′ of a purine nucleoside.

19. The oligomeric compound of any of claims 1-18, wherein each purine in the central region is linked to an adjacent nucleoside by a methanesulfonylphosphoramidate internucleoside linkage at the 3′-side of the purine nucleoside, the 5′-side of the purine nucleoside, or both the 3′-side and the 5′-side of the purine nucleoside, independently for each purine nucleoside in the central region.

20. The oligomeric compound of any of claims 1-19, wherein each internucleoside linkage within the central region is selected from a methanesulfonylphosphoramidate internucleoside linkage and a phosphorothioate internucleoside linkage.

21. The oligomeric compound of any of claims 1-20, wherein each internucleoside linkage within the 5′-region is selected from a phosphodiester internucleoside linkage and a phosphorothioate internucleoside linkage.

22. The oligomeric compound of any of claims 1-21, wherein each internucleoside linkage within the 3′-region is selected from a phosphodiester internucleoside linkage and a phosphorothioate internucleoside linkage.

23. The oligomeric compound of any of claims 1-22, wherein the internucleoside linkage between the 5′-region and the central region is selected from a phosphodiester internucleoside linkage and a phosphorothioate internucleoside linkage.

24. The oligomeric compound of any of claims 1-23, wherein the internucleoside linkage between the 5′-region and the central region is a phosphodiester internucleoside linkage.

25. The oligomeric compound of any of claims 1-23, wherein the internucleoside linkage between the 5′-region and the central region is a phosphorothioate internucleoside linkage.

26. The oligomeric compound of any of claims 1-25, wherein the internucleoside linkage between the 3′-region and the central region is selected from a phosphodiester internucleoside linkage and a phosphorothioate internucleoside linkage.

27. The oligomeric compound of any of claims 1-25, wherein the internucleoside linkage between the 3′-region and the central region is a phosphodiester internucleoside linkage.

28. The oligomeric compound of any of claims 1-25, wherein the internucleoside linkage between the 3′-region and the central region is a phosphorothioate internucleoside linkage.

29. The oligomeric compound of any of claims 1-28, wherein each internucleoside linkage of the modified oligonucleotide is selected from among a methanesulfonyl phosphoramidate, a phosphodiester and a phosphorothioate internucleoside linkage.

30. The oligomeric compound of any of claims 1-29, comprising an internucleoside linkage of Formula IV.

31. The oligomeric compound of any one of claims 1-30, wherein each 5′-region nucleoside comprises a modified sugar moiety.

32. The oligomeric compound any of claims 1-31, wherein each 3′-region nucleoside comprises a modified sugar moiety.

33. The oligomeric compound of any of claims 1-32, wherein each central region nucleoside is a deoxynucleoside.

34. The oligomeric compound of any of claims 1-33, wherein each deoxynucleoside comprises a 2′-β-D-deoxyribosyl sugar moiety.

35. The oligomeric compound of any of claims 1-34, wherein at least one 5′-region nucleoside and/or at least one 3′-region nucleoside comprises a bicyclic sugar moiety.

36. The oligomeric compound of claim 35, wherein each bicyclic sugar moiety is selected from cEt and LNA.

37. The oligomeric compound of any of claims 1-36, wherein at least one 5′-region nucleoside and/or at least one 3′-region nucleoside comprises a 2′-substituted sugar moiety.

38. The oligomeric compound of any of claims 1-32 or 34-37, wherein at least one central region nucleoside comprises a 2′-substituted sugar moiety.

39. The oligomeric compound of any of claims 37-38 wherein the 2′-substituent of each 2′-substituted sugar moiety is selected from OCH₃, and O(CH₂)₂—OCH₃.

40. The oligomeric compound of any of claims 1-39, wherein the modified oligonucleotide comprises at least one modified nucleoside comprising a modified nucleobase.

41. The oligomeric compound of claim 40, wherein the modified nucleobase is 5-methyl cytosine.

42. The oligomeric compound of any of claims 1-41, comprising a conjugate group.

43. The oligomeric compound of claim 42, wherein the conjugate group comprises a cell targeting moiety.

44. The oligomeric compound of any of claims 1-43, wherein 2 to 5 of the internucleoside linkages within the central region are sulfonylphosphoramidate internucleoside linkages.

45. The oligomeric compound of any of claims 1-43, wherein exactly two of the internucleoside linkages within the central region are sulfonylphosphoramidate internucleoside linkages.

46. The oligomeric compound of any of claims 1-43, wherein exactly three of the internucleoside linkages within the central region are sulfonylphosphoramidate internucleoside linkages.

47. The oligomeric compound of any of claims 1-43, wherein exactly four of the internucleoside linkages within the central region are sulfonylphosphoramidate internucleoside linkages.

48. The oligomeric compound of any of claims 1-43, wherein exactly five of the internucleoside linkages within the central region are sulfonylphosphoramidate internucleoside linkages.

49. The oligomeric compound of any of claims 1-48, wherein the 5′-region is at the 5′ terminal end of the modified oligonucleotide and the 3′-region is at the 3′ terminal end of the modified oligonucleotide.

50. A method comprising administering an oligomeric compound of any of claims 1-49 to an animal.

51. The method of claim 50 wherein at least two doses of the oligomeric compound of any of claims 1-49 are administered to an animal and the oligomeric compound is administered to the animal at a dose frequency of once per 4 months, 5 months, 6 months, 7 months, 8 months, 9 months, 10 months, 11 months, or a year or more than a year.

52. The method of claim 50 or 51, wherein the oligomeric compound is dosed at an amount of 50 mg to 500 mg.

53. The method of claim 50 or 51, wherein the oligomeric compound is administered systemically.

54. The method of claim 50-53, wherein the oligomeric compound is administered subcutaneously.
